(19) United States
(12) Patent Application Publication
(10) Pub. No.: US 2010/0310504 A1
(43) Pub. Date: Dec. 9, 2010

(54) METHODS FOR TREATING FIBROSIS BY MODULATING CELLULAR SENESCENCE

(76) Inventors: Scott W. Lowe, Cold Spring Harbor, NY (US); Valery Krizhanovsky, Huntington Station, NY (US); Lars Zender, Hannover (DE)

Correspondence Address: WilmerHale/Cold Spring Harbor Laboratory, 399 Park Avenue, New York, NY, 10022, US

(21) Appl. No.: 12/679,835

(22) PCT Filed: Sep. 25, 2008
(86) PCT No.: PCT/US2008/077732

§ 371 Date: Jul. 14, 2010

Related U.S. Application Data
(60) Provisional application No. 60/995,647, filed on Sep. 26, 2007.
(60) Provisional application No. 61/091,328, filed on Aug. 22, 2008.

(51) Int. Cl. A61K 38/20; A61K 38/21; A61K 38/18; A61K 35/12; A61K 31/7052; A61K 39/395; C12Q 1/02; A61P 1/16

(52) U.S. Cl. 424/85.2; 424/85.7; 424/85.5; 514/8.8; 514/8.9; 424/85.1; 424/93.71; 514/44 A; 424/172.1; 435/29

(57) ABSTRACT

Fibrosis arises as part of a wound healing response that maintains organ integrity following catastrophic tissue damage, but can also contribute to a variety of human pathologies, including liver cirrhosis. The invention demonstrates that cellular senescence acts to limit the fibrogenic response to tissue damage, thereby establishing a role for the senescence program in pathophysiological settings beyond cancer. Accordingly, the methods of the invention relate to modulating cellular senescence in disease tissue that have elevated numbers of senescent cells, such as in fibrotic tissues.

This application claims the benefit of priority of U.S. Provisional Application Ser. No. 60/995,647, filed Sep. 26, 2007, and U.S. Provisional Application Ser. No. 61/091,328, filed Aug. 22, 2008, the disclosures of which are hereby incorporated by reference in their entirety.

This invention was made with government support under grant No. AG16379 awarded by the National Institutes of Health. The United States government has certain rights in this invention.

### 1. BACKGROUND

Cellular senescence is a stable form of cell cycle arrest that may limit the proliferative potential of pre-malignant cells. Initially defined by the phenotype of human fibroblasts undergoing replicative exhaustion in culture, senescence can be triggered in many cell types in response to diverse forms of cellular damage or stress. Although once considered a tissue culture phenomenon, recent studies demonstrate that cellular senescence imposes a potent barrier to tumorigenesis and contributes to the cytotoxicity of certain anticancer agents. Senescent cells have also been observed in certain aged or damaged tissues. However, the functional contribution of cellular senescence to non-cancer pathologies has not been examined.

Although senescent cells can remain viable in culture indefinitely, their fate in tissue is not well characterized. On one hand, benign melanocytic nevi (moles) are highly enriched for senescent cells yet can exist in skin throughout a lifetime, implying that senescent cells can be stably incorporated into tissue. On the other hand, liver carcinoma cells induced to undergo senescence in vivo can be cleared by components of the innate immune system leading to tumor regression (Xue, W. et al., (2007) Nature 445, 656-660; which is hereby incorporated by reference in its entirety). Therefore, in some circumstances, senescent cells can turn over in vivo.

Liver cirrhosis is a major health problem worldwide, and the 12th most common cause of death in the United States. Liver fibrosis acts as a precursor to cirrhosis and is triggered by chronic liver damage produced by hepatitis virus infection, alcohol abuse, or nonalcoholic steatohepatitis (NASH, fatty liver disease). The hepatic stellate cell (HSC, also called Ito cell) is a key cell type that contributes to liver fibrosis. Upon liver damage, HSCs become “activated”—i.e. they differentiate into myofibroblasts, proliferate and produce the network of extracellular matrix that is the hallmark of the fibrotic scar. Following acute damage activated HSCs probably support hepatocyte proliferation and organ repair; however, during chronic damage the excessive extracellular matrix produced by these cells disrupts liver cytoarchitecture leading eventually to cirrhosis and liver failure. Others have reported that Natural Killer (NK) cell mediated killing of activated stellate cells can help to ameliorate liver fibrosis (Radaeva, S. et al., (2006) Gastroenterology, 130:435-452; and Friedman US20070197424). However, these reports do not disclose that the normal fibrosis resolution involves senescence of the activated stellate cells and killing/clearance of the senescent stellate cells by NK cells and the innate immune system.

SA-β-gal positive cells have been observed in cirrhotic livers of human patients (Wiemann, S. et al., (2002) Faseb J 16, 935-942; which is hereby incorporated by reference in its entirety, including the disclosure relating to SA-β-gal protocols), although these putative senescent cells were suggested to be adult hepatocytes. The functional contribution of cellular senescence to non-cancer pathologies has not previously been examined. Herein, the disclosure provides the finding that cellular senescence limits fibrosis and that removal or killing of senescent cells by cells of the innate immune system helps to resolve fibrosis. Thus, the invention includes methods that reverse, prevent, or limit fibrosis by modulating the senescence of cells that contribute to or cause fibrosis. Further, the invention provides methods to screen for anti-fibrotic agents by screening for agents that can promote the association of innate immune cells and senescent cells. Methods that aim to treat fibrosis by targeting senescent cells is preferred over prior art methods because methods that only target events upstream of senescence, such as killing activated stellate cells, can work against the normal processes of tissue healing.

### 2. SUMMARY OF THE INVENTION

Cellular senescence acts as a potent mechanism of tumor suppression; however, its functional contribution to non-cancer pathologies has not been examined. Here, it is shown that senescent cells accumulate in murine livers treated to produce fibrosis, a precursor pathology to cirrhosis. The senescent cells are derived primarily from activated hepatic stellate cells, which initially proliferate in response to liver damage and produce the extracellular matrix deposited in the fibrotic scar. In mice lacking key senescence regulators, stellate cells continue to proliferate, leading to excessive liver fibrosis. Furthermore, senescent activated stellate cells exhibit a gene expression profile consistent with cell cycle exit, reduced secretion of extracellular matrix components, enhanced secretion of extracellular matrix degrading enzymes, and enhanced immune surveillance. Natural killer cells preferentially kill senescent activated stellate cells in vitro and in vivo, thereby facilitating the resolution of fibrosis. Therefore, the senescence program, which comprises the promotion of senescence in cells that cause fibrotic tissue accumulation or scars and the resolution of senescent cells by the killing and removal of the cells, limits the fibrogenic response to acute tissue damage.

Thus, in various aspects, the invention provides methods for treating (which includes limiting, reversing, inhibiting, resolving) fibrosis in a tissue of a subject, the method comprising modulating senescence by increasing or promoting the senescence of the cells contributing to fibrosis in the tissue. In another aspect, methods for treating fibrosis in a tissue of a subject comprises modulating senescence by increasing the killing or removal of senescent cells in the fibrotic tissue (not necessarily only those senescent cells that were contributing to the fibrosis prior to their senescence). In another aspect, the methods for treating fibrosis comprise both the steps of promoting senescence in the fibrotic tissue and killing and/or clearing the senescent cells in the fibrotic tissue. In one aspect, the cells contributing to fibrosis in the tissue are myofibroblasts, myofibroblast-like cells (i.e., activated hepatic stellate cells), or fibroblasts that are producing the extracellular matrix that is part of the fibrotic scar. The fibrotic tissue to be treated can be, for example, skin, liver, lung, atherosclerotic tissue, pancreas, or prostate. Fibrotic tissues for potential treatment can be determined by assaying tissues or cells from animal models (or samples/biopsies from humans) of disease or injury for an increased number of senescent cells, which assay can comprise staining for an increase in SA-β-Gal positive cells as compared to controls.

In one aspect, the invention provides a method for treating fibrosis in a subject comprising modulating the amount of senescent cells in the fibrotic tissue by increasing the number of innate immune system cells in the fibrotic tissue to an amount sufficient to increase the killing of senescent cells in the fibrotic tissue. Exemplary innate immune system cells include but are not limited to mast cells, phagocytes (such as macrophages, neutrophils, and dendritic cells), basophils, eosinophils, natural killer cells, natural killer T-cells, and gamma-delta T-cells. Increasing the number of innate immune system cells in the fibrotic tissue can be accomplished by administering to the subject one or more chemical compounds and/or proteins capable of activating and/or recruiting innate immune system cells to the fibrotic tissue. Exemplary chemical compounds or proteins capable of activating and/or recruiting innate immune system cells include, but are not limited to, IFN-α, IFN-γ, IL-1, IL-2, IL-6, IL-8, IL-12, IL-13, IL-15, IL-18, IL-24, BMP2, GDF15, CXCL1, CXCL2, CXCL3, CXCL5, CXCL12, CCL20, CCL15, CCL26, LIF, CNTF, BSF3, CTF1, MCP-1, Polyl:C, an agonist of NKp30, an agonist of NKp44, an agonist of NKp46, an agonist of an NKG2D receptor, an agonist of a SLAM-related receptors (SRR), and an agonist of CD48.

In one aspect, the invention provides a method for treating fibrosis in a subject, the method comprising administering to the subject one or more agents in an amount sufficient to cause an increase in the number of activated innate immune cells in the fibrotic tissue and an increase in the killing of senescent cells in the fibrotic tissue. As used herein, the term “agent” includes any small molecule chemical compound, protein, peptide, nucleic acid, toxin, or other substance that can promote the activation of an innate immune cell, the recruitment of an innate immune cell, senescence within a cell, the killing of senescent cells, etc. The fibrotic tissue can be, for example, in the liver, lung, atherosclerotic tissue, skin, pancreas, or prostate of the subject.

In one aspect, the invention provides a method for treating fibrosis in a subject, where the method comprises: (a) administering to the subject one or more agents that promotes the senescence of myofibroblasts in the fibrotic tissue, and (b) administering to the subject one or more agents that promotes the killing of the senescent myofibroblasts in the fibrotic tissue. In one aspect, the agent that promotes the senescence of myofibroblasts in the fibrotic tissue comprises an expression vector that encodes p53, p21Cip1/Waf1 cyclin-dependent kinase inhibitor, or an miR-34 class of microRNA. The expression vector can be, for example, based on an alpha virus, an adeno-associated virus, or a retrovirus. The expression vector can be contained within the recombinant genome or transgene in a retroviral virion which is administered to a subject. In one aspect, the fibrosis occurs in the liver of the subject, and the expression vector comprises a GFAP promoter.

In another aspect, the agent(s) that promotes the senescence of myofibroblasts in the fibrotic tissue comprises an expression vector that codes for a dsRNA or a short-hairpin RNA molecule that can cause post-transcriptional silencing of cyclin-dependent kinases 2 and/or 4 via RNA interference.

In another aspect, the agent(s) that promotes the killing of senescent myofibroblasts comprises an immunostimulatory molecule capable of activating and/or recruiting an innate immune system cell in/to the fibrotic tissue. In one aspect, such agent(s) comprise an immunostimulatory molecule capable of activating NK cells and/or recruiting NK cells to the fibrotic tissue. In another aspect, agents capable of activating NK cells and/or recruiting NK cells include, but are not limited to, an agonist of NKp30, NKp44, NKp46, NKG2D receptors, or an agonist of SLAM-related receptors (SRR).

In other aspects, the invention provides a method for treating fibrosis in a subject, comprising increasing the killing of senescent cells in the fibrotic tissue of the subject by administering to the subject an antibody that targets one or more cell surface proteins upregulated or differentially expressed on the senescent cells as compared to their activated precursor state. Exemplary upregulated or differentially expressed cell surface protein(s) on senescent cells as compared to their precursors include, but are not limited to, ligands of NK activation receptors (including ligands of NKp30, NKp44, NKp46, NKG2D receptors) ULBP2, PVR, and CD58. In one aspect, a ligand of NKG2D receptor is MICA.

In one aspect, the invention provides a method for treating fibrosis in the liver of a subject comprising modulating senescence in the liver by increasing the number of innate immune system cells in the liver to an amount sufficient to increase the killing of senescent activated hepatic stellate cells in the liver. In one aspect, this method comprises increasing the number of NK cells in the liver to an amount sufficient to increase the killing of senescent activated hepatic stellate cells. In one aspect, increasing the number of NK cells in the liver can comprise treatment with one or more of interferon-gamma, Polyl:C, an agonist of NKp30, an agonist of NKp44, an agonist of NKp46, an agonist of an NKG2D receptor, an agonist of a SLAM-related receptors (SRR), and an agonist of CD48.

In one aspect, the invention provides a method for treating liver fibrosis, the method comprising: (a) increasing the senescence of activated hepatic stellate cells in liver, and (b) increasing the killing of senescent activated hepatic stellate cells.

In another aspect, increasing the number of NK cells in the liver comprises isolating peripheral blood from the subject, expanding NK cells from the peripheral blood in culture, and administering the expanded population of NK cells back to the subject. In one aspect, the expanded population of NK cells are administered to the spleen of the subject such that the NK cells migrate more readily to the liver.

In another aspect, the invention provides a method for treating fibrosis in a subject, the method comprising administering to the subject allogeneic NK cells, which are activated and expanded ex vivo in an amount sufficient to cause an increase in the killing of senescent cells in the fibrotic tissue. The allogeneic NK cells can comprise peripheral blood NK cells from the subject itself or from a compatible donor.

In one aspect, the invention provides a method for treating fibrosis in a subject, comprising increasing the killing of senescent cells in the fibrotic tissue of the subject by administering to the subject an antibody that targets one or more cell surface proteins upregulated or differentially expressed on the senescent cells as compared to their activated precursor state. Cell surface proteins upregulated or differentially expressed on senescent cells that can serve as antibody target antigens include for example CD58, MICA (MHC class I related protein A), ULBP2(UL16 binding protein 2), and PVR(CD155/Poliovirus receptor). In one aspect, the antibody is bivalent and targets two cell surface proteins upregulated or differentially expressed on senescent cells.

In one aspect, the invention provides a method for treating liver fibrosis in a subject, comprising increasing the killing of senescent activated hepatic stellate cells in the liver by administering to the subject a liposome that targets senescent activated hepatic stellate cells. The liposome that targets such cells can be coated with ligands that bind to cell-surface proteins that are upregulated on senescent cells, such as CD58, MICA (MHC class I related protein A), ULBP2(UL16 binding protein 2), and PVR(CD155/Poliovirus receptor). The liposome can contain within it chemical compounds that cause the death of the senescent cell.

In another aspect, liposomes that target HSC cells can be used to promote senescence. In one aspect, the liposome can be used as a carrier for an expression vector, which can be used to express p53, p21/Cip1/Waf1 cyclin-dependent kinase inhibitor, p16INK4a, or miR-34 class of microRNAs in the HSC cell to promote senescence. In another aspect, the liposome can be used to deliver an expression vector that expresses a siRNA or shRNA molecule that suppresses expression of cyclin-dependent kinase 2 or cyclin-dependent kinase 4.

In other aspects, the methods comprise the combination of increasing the number of innate immune system cells in the fibrotic tissue and administering an antibody or antibodies that target one or more upregulated or differentially expressed cell surface proteins on the senescent cell in the fibrotic tissue. In some aspects, the antibodies comprise constant regions capable of binding to Fc-Receptors expressed by innate immune cells. Thus, by coating senescent cells with antibodies, this will help to increase senescent cell killing and/or clearance via Fc-receptor mediated mechanisms.

In one aspect, the invention provides co-administration methods, where any of the therapeutic methods disclosed herein are used in combination with the administration of antifibrotic compounds such as colchicine, pentoxifylline, halofuginone, prolyl 4-hydroxylaseinhibitors such as HOE 077 or S4682, serine protease inhibitors such as camostat mesilate dilinoleoyl-phophatidylcholine, PPARγ antagonists such as rosiglitazone, angiotesin II receptor inhibitors such as losartan, cariporide, gliotoxin, α-tocopherol, S-adenosyl-methionine, Sho-saiko-to, and quercetin.

In other aspects, the invention provides the use of compounds that can promote the increase of innate immunity cells to fibrotic tissue for the manufacture of a medicament for treating or limiting fibrosis.

In other aspects, the invention provides the use of an antibody that can promote the killing of senescent cells in fibrotic tissue for the manufacture of a medicament for treating or limiting fibrosis.

In another aspect, the invention provides a method for selecting compounds that have the potential to limit fibrosis, the method comprising testing whether a compound can promote the association between an NK cell and a senescent HSC cell. In another aspect, the method comprises testing whether a compound can promote a direct effect on killing—for example making senescent cells more susceptible to NK-cell mediated killing.

In one aspect, the invention provides a method of screening for a compound for treating fibrosis, the method comprising: (a) providing a culture, which culture comprises myofibroblast (or myofibroblast-like) cells that are growing, senescent myofibroblast (or myofibroblast-like) cells, and NK cells; and (b) testing whether the addition of a compound causes a specific increase in the death of senescent myofibroblast cells, wherein the increase in the death of senescent cells is not specific if the addition of the compound also causes an increase in the death of growing myofibroblast cells and/or an increase in the death of NK cells. In another aspect, step (b) can further comprise testing whether the addition of the compound causes a specific increase in the death of senescent cells that is NK-cell dependent, wherein an increase in the death of senescent cells is not NK-cell dependent if the addition of the compound causes a specific increase in the death of senescent cells in a culture that does not contain NK cells.

### 3. BRIEF DESCRIPTION OF THE DRAWINGS

The corresponding color-versions of the Figures below along with their legends from Krizhanovsky, V. et al., “Senescence of Activated Stellate Cells Limits Liver Fibrosis,” Cell, 134, 657-667 (Aug. 22, 2008), are hereby incorporated by reference.

FIG. 1. Senescent cells are present in fibrotic livers. FIG. 1A: CCl4 (Fibrotic) but not vehicle (control) treated livers exhibit fibrotic scars (evaluated by H&E and Sirius Red staining). Multiple cells in the areas around the scar stain positively for senescence markers (SA-β-gal and p16 staining). FIG. 1B: The cells around the scar also co-express senescence markers p21, p53 and Hmga1, and are distinct from proliferating Ki67 positive cells. Numbers in the lower left corner indicate number of double positive cells (yellow in the color figure) out of p21 positive cells (green in the color figure). Scale bars are 50 μm.

FIG. 2. Senescent cells are derived from activated HSCs. FIG. 2A: Senescent cells, identified by p53 and Hmga1 positive staining, express activated HSC markers Desmin and αSMA. Upper panels: Hmga1 positive nuclei (red arrows), and Desmin cytoplasmic staining (green arrows) in same cells. Lower panels: p53 positive nuclei (green arrows) and αSMA (red arrows) cytoplasmic staining in same cells. FIG. 2B. Senescent cells, identified by SA-β-gal stain positive for HSC marker αSMA on serial sections of mouse fibrotic liver. FIG. 2C: Senescent cells, identified by p21 or p16 stain positive for HSC marker αSMA on serial sections of human fibrotic liver. p21 and p16 positive cells are not present in normal liver sections.

FIG. 3. Intact senescence pathways are required to restrict fibrosis progression. FIG. 3A: Mice lacking p53 develop pronounced fibrosis following CCl4 treatment, as identified by Sirius Red staining Livers from wt or p53^{-/-} mice treated with CCl4 were harvested and subjected to Sirius Red and SA-β-gal staining, and p16 immunocytochemistry and p53 immunofluorescence analysis. There are fewer senescent cells in mutant livers, as identified by SA-β-gal activity. FIG. 3B: Quantification of fibrosis based on Sirius Red staining Values are means +SE. Fibrotic area in mutant animals was compared to wild type (wt) of corresponding time point using Student's t-test (*−p<0.05, **−p<0.01). FIG. 3C: Immunoblot showing expression of αSMA in liver of mice treated with CCl4. There are more activated HSCs in the p53 and INK4a/ARF mutant mice than in wild type as shown by higher protein expression of the activated HSC marker αSMA analyzed by immunoblot. Two upper panels represent different exposures times for αSMA. FIG. 3D. BrdU incorporation over 2 hours in activated HSCs derived from wt and DKO mice. FIG. 3E. SA-β-gal activity and fibrosis (evaluated by Sirius Red) in livers from wt and p53−/−;INK4a/ARF−/− (DKO) mice treated with CCl4. Scale bars are 100 μm. FIG. 3F: Fibrosis was quantified as described before. There is stronger fibrosis in mice lacking both p53 and INK4a/ARF. FIG. 3G: Expression of αSMA in wild type and DKO fibrotic livers was evaluated by immunoblot. FIG. 3H: Fibrosis in TRE-shp53 (Tg) and GFAP-tTA;TRE-shp53 (DTg) was quantified as described before. FIG. 31: Expression of αSMA in Tg and DTg fibrotic livers was evaluated by immunoblotting. FIG. 3J: There are more proliferating activated HSCs (Ki67 and αSMA positive) in DTg livers derived from mice treated with CCl4.

FIG. 4. An intact senescence response promotes fibrosis resolution. Mice were treated with CCl4 for 6 weeks and livers were harvested 10 and 20 days following cessation of the treatment. FIG. 4A: There is a significant retention of fibrotic tissue in p53−/− livers compared to wild-type (wt) livers as identified by Sirius Red staining at the 10 and 20 days time-points. SA-β-gal staining shows senescent cells at fibrotic liver, 10 and 20 days following cessation of fibrogenic treatment. Senescent cells are eliminated from the liver during reversion of fibrosis. Quantification of fibrosis in wt and p53−/− (FIG. 4B), or wt and p53−/−;INK4a/ARF−/− (DKO) (FIG. 4C), mice based on Sirius Red staining of livers. Values are means +SE; fibrotic area in mutant animals was compared to wt of corresponding time point using Student's t-test (*−p<0.05, **−p<0.01, ***−p<0.001).

FIG. 5. Senescent activated HSCs downregulate extracellular matrix production and upregulate genes that modulate immune surveillance. FIG. 5A: Activated HSCs treated with a DNA damaging agent, etoposide (Senescent), and intact proliferating cells (Growing) were stained for SA-β-gal activity and for expression of HSC markers (αSMA, GFAP, Vimentin) by immunofluorescent staining (green) and counterstained with DAPI (blue). Insets: Higher magnification of DAPI stained nuclear DNA shows presence of heterochromatic foci in senescent cells. Arrowheads point to nuclei shown in the insets. FIG. 5B: Quantitative RT-PCR analysis reveals decreased expression of extracellular matrix components in senescent activated HSCs. Values are means +SE. FIG. 5C. Extracellular matrix degrading matrix metalloproteinases are upregulated in senescent activated HSCs. Values represent the average of duplicate samples from microarrays. FIG. 5D. Quantitative RT-PCR analysis reveals increased expression of cytokines, adhesion molecules and NK cell receptor ligands in senescent activated HSCs and IMR-90 cells as compared to growing cells. Values are means +SE.

FIG. 6. Immune cells recognize senescent cells. FIG. 6A: Immune cells are adjacent to activated HSCs in vivo as identified by electron microscopy of normal and fibrotic mouse livers. Immune cells (lp—lymphocytes, mφ—macrophage, np—neutrophil) localize adjacent to activated HSC. Scale bar is 5 μm. FIG. 6B: Immune cells identified by CD45R (CD45) reside in close proximity to senescent cells (identified by p21, p53 and Hmga1) in mouse fibrotic liver. FIGS. 6C, 6D: Senescent can be recognized by immune cells in vitro. Images from time lapse microscopy of the same field at start (0) and 10 hours after presenting interaction between NK cells (uncolored) and growing (C) or senescent (D) IMR-90 (pseudocolored, green) cells. Original images and time points are presented in FIG. 11. Scale bar is 100 μm. FIGS. 6E, 6F: Human NK cell line, YT, exhibits preferential cytotoxicity in vitro towards senescent activated HSCs (E) or senescent IMR-90 cells (F) compared to growing cells. In IMR-90 cells senescence was induced by DNA damage, extensive passaging in culture or by infection with oncogenic rasV12. Both uninfected and empty vector infected growing cells were used as controls. At least three independent experiments were performed in duplicates. Cytotoxicity based on crystal violet quantification at OD595 are shown, values are means +SE, **−p<0.005 using Student's t-test.

FIG. 7. NK cells participate in fibrosis reversion and senescent cell clearance in vivo. FIG. 7A. Wild type mice treated with CCl4 were treated with either an anti-NK antibody (to deplete NK cells), polyI:C (as an interferon-γ activator) or saline (as a control) for 10 or 20 days prior to liver harvest. Liver sections stained for SA-β-gal show positive cells are retained in fibrotic livers following depletion of NK cells upon treatment with an anti-NK antibody in mice. In contrast, treatment with polyI:C results in enhanced clearance of senescent cells. FIG. 7B. Fibrotic tissue is retained upon depletion of NK cells as visualized by Sirius Red staining in contrast to saline or polyI:C treated mice, where it was depleted more efficiently. FIG. 7C. Quantification of fibrosis based on Sirius Red staining following 10 or 20 days of treatment with either saline, anti-NK antibody or Polyl:C. Values are means +SE. Fibrotic area in anti-NK or polyI:C treated animals was compared to saline treated animals of corresponding time point using Student's t-test (*−p<0.05, **−p<0.01). FIGS. 7D, 7E. Expression of αSMA in fibrotic livers after 10 days treatment with anti-NK antibody was increased comparing to saline treated ones, while its expression was decreased in polyI:C treated mice as evaluated by quantitative RT-PCR analysis (D) and immunoblot (E).

FIG. 8. Quantitative RT-PCR analysis of expression of a stellate cell marker αSMA (Acta2) and a fibrosis molecular marker Tgfβ₁ reveals increased expression of these genes in fibrotic livers of p53 mutant animals relative to wild type. This difference persists 10 and 20 days following cessation of fibrogenic treatment in p53 mutant animals.

FIG. 9. Proliferating cells (Ki67 positive, green) are abundant 10 days after cessation of fibrogenic treatment in p53^{-/-} livers, but not wild type liver.

FIG. 10. p53^{-/-};INK4a/ARF^{-/-} activated HSCs bypass senescence in culture. HSCs were prepared from wild type and p53^{-/-};INK4a/ARF^{-/-} (double knock out “DKO”) mouse livers. Following 3 weeks in culture, cells from both genotypes express activated HSC marker, αSMA. Wild type cells stop proliferating and exhibit a flattened senescence-like morphology, while DKO cells continue to proliferate.

FIG. 11. p53^{-/-};INK4a/ARF^{-/-} (DKO) mice accumulate excessive ascites fluid. Wild type (wt) and p53^{-/-};INK4a/ARF^{-/-} (DKO) animals were treated with CCl4 for 6 weeks. The animals were imaged (representative picture) and abdominal width measured and presented as mean +SE (right panel, ***−p<0.001).

FIG. 12. Stellate cell specific p53 knock-down in GFAP-tTA and TRE-shp53 transgenic animals leads to activated HSC expansion in vivo. FIG. 12A: RT-PCR with tTA specific primers shows tTA expression in the liver of GFAP-tTA and GFAP-tTA;TRE-shp53 mice, but none in TRE-shp53 animals. FIG. 12B: Quantitative RT-PCR for microRNA of shp53 in the livers from GFAP-tTA, TRE-shp53 and GFAP-tTA;TRE-shp53 mice reveals expression of the microRNA only in GFAP-tTA;TRE-shp53 mice. FIG. 12C: There are more proliferating activated HSCs in GFAP-tTA;TRE-shp53 livers following CCl4 treatment, than in the TRE-shp53 and GFAP-tTA;TRE-shp53 mouse livers as revealed by immunofluorescence analysis of a proliferation marker Ki67, and activated HSC marker, αSMA. Lower panel shows only Ki67 signal of corresponding upper panel.

FIG. 13. There are more activated HSCs in p53^{-/-};INK4a/ARF^{-/-} (DKO) than in wild type (wt) livers following reversion of fibrosis as revealed by immunofluorescence analysis.

FIG. 14. Extracellular matrix components are downregulated in senescent activated HSCs as assayed by gene expression microarray analysis of human activated HSCs. Values represent the average of duplicate samples.

FIG. 15. Diagram of KEGG Cytokine-Cytokine receptor interaction pathway. Genes, up-regulated in senescent activated HSCs are circled.

FIG. 16. Time lapse microscopy of the same field up to 10 hours after interaction between NK cells and growing or senescent IMR-90 cells. Time indicated in the upper left corner of each image. Scale bar is 100 um.

FIG. 17. Activated AKT is expressed in activated HSCs in vivo and in cultured cells. FIG. 17A: pAKT(473) is expressed in a subset of activated HSC (αSMA positive, green) in fibrotic livers as analyzed by immunofluorescence. FIG. 17B. pAKT(473) is expressed in a subset of human activated HSC in culture at passage 9 as was analyzed by immunofluorescence.

FIG. 18. Proposed model: senescence of activated HSC acts as a coordinated program to limit fibrosis. Senescence of stellate cells limits fibrosis by executing the coordinated program characterized by cell cycle exit, down-regulation of extracellular matrix components, upregulation of extracellular matrix degrading enzymes and enhanced immunosurveillance. This proposed model is applicable to other tissues with fibrosis.

FIG. 19. SA-β-gal staining on tissue from fibrotic lung. The staining shows that senescent cells are present in the fibrotic lung.

FIG. 20. Perforin block prevents killing of senescent cells by NK cells.

FIG. 21. Prfl^{-/-} mice develop stronger fibrosis. Upper panel: Sirius red staining of fibrotic liver sections WT and Prfl^{-/-} mice. Lower panel left: Evaluation of fibrotic area indicates significantly stronger fibrosis in Prfl^{-/-} mice. Lower panel right: Western blot analysis shows higher expression of αSMA and p21 in the livers of Prfl^{-/-} mice.

### 4. DETAILED DESCRIPTION OF THE INVENTION

Fibrosis arises as part of a wound healing response that maintains organ integrity following catastrophic tissue damage, but can also contribute to a variety of human pathologies, including liver cirrhosis. To study the role of senescence in fibrosis, a murine model system was used where fibrosis of the liver was induced by treating mice with CCl4. As presented in the Examples, it is shown that senescent cells in fibrotic livers of CCl4 treated mice arise from activated stellate cells—a cell type that initially proliferates in response to hepatocyte cell death and is responsible for the extracellular matrix production that is the hallmark of the fibrotic scar. Surprisingly, the senescence of activated HSCs limits the accumulation of fibrotic tissue following chronic liver damage, and facilitates the resolution of fibrosis upon withdrawal of the damaging agent. Thus, it is demonstrated that cellular senescence acts to limit the fibrogenic response to tissue damage, thereby establishing a role for the senescence program in pathophysiological settings beyond cancer. Accordingly, the methods of the invention relate to modulating cellular senescence in disease tissue that have elevated numbers of senescent cells, such as in fibrotic tissues.

The disclosure provides the finding that NK cells preferentially associate with senescent activated HSCs. Thus, using this finding, the invention provides methods of screening for compounds that can promote or enhance NK cell (or other innate immunity cell) association with senescent activated HSCs. This screening method can be varied by focusing on specific functional associations, such as cell killing, disruption in particular ligand-receptor interactions, etc.

Although methods that eliminate activated HSCs might reduce fibrosis, such methods are often less preferred due to one or both of the following reasons: (1) activated HSCs play a positive role in response to acute injury, (2) targeting activated HSCs does not necessarily remove senescent activated HSCs, whose clearance is important to complete healing and prevent possible tissue destruction and/or cancer promoting effects from the accumulation of senescent cells. Thus, in some embodiments, preferred methods for limiting fibrosis comprise the promotion of senescence and/or the specific killing and/or clearance of senescent cells as opposed to killing their activated precursors.

### 4.1 Cellular Senescence and Methods for Determining Senescence Related Pathologies

In various embodiments, the invention seeks to treat fibrosis by modulating cellular senescence in damaged or diseased tissue. As used herein, “modulating” senescence refers to affecting some aspect of the senescence program or machinery within the cell or affecting the senescent cell itself For example, modulating senescence includes triggering senescence in a cell, killing a senescent cell, and/or clearing a senescent cell. In some embodiments, the methods for treating fibrosis comprise at least the step of promoting senescence of myofibroblasts or extracellular-matrix producing cells or cells that contribute to the formation of fibrotic scars. As used herein “myofibroblasts” includes myofibroblast-like cells, such as activated hepatic stellate cells. In some embodiments, the methods for treating fibrosis comprise at least the step of stimulating the innate immune system in the subject such that senescent cells in the fibrotic tissue are more rapidly and effectively killed/cleared. By preventing the accumulation of senescent cells, the present methods seek to help resolve fibrosis and also to prevent the progression from fibrosis to cancer. Senescence cells are cleared to complete healing and prevent possible tissue destruction and/or cancer promoting effects from the accumulation of senescent cells. To determine which pathologies can be treated by the methods, cells or tissues isolated from fibrotic tissue from human subjects or animal models can be assayed for an increase/accumulation of senescent cells.

Senescent cells display a large flattened morphology and accumulate a senescence-associated β-galactosidase (SA-β-gal) activity that distinguishes them from most quiescent cells (Campisi, J., and d'Adda di Fagagna, F. (2007), Nat Rev Mol Cell Biol 8, 729-740; incorporated herein by reference in its entirety including the disclosure relating to SA-β-gal). β-galactosidase, a lysosomal hydrolase, is normally active at pH 4, but often in senescent cells β-galactosidase is active at pH 6. Thus, for example, one method to determine whether senescence might play a functional role in the pathology of a disease is to assay whether the disease tissue stains positively for SA-β-Gal. For example, SA-β-Gal positive cells can be found in damaged or diseased or aging tissue, such as in skin, atherosclerotic plaque, pancreas, prostate, lung fibrosis, and liver fibrosis and cirrhosis.

Senescent cells also display abnormal genetic features. Normal human cells are diploid, which means they have two copies of each chromosome. Yet with each subcultivation, the percentage of polyploid cells—i.e., with three or more copies of chromosomes—increases. Mutations to the mitochondrial DNA (mtDNA) also appear to increase with age in vivo, though at low levels. For example, the first identified mutation was a deletion of 4,977 base pairs (bp) in the 16,569 by mtDNA. This deletion is observed both in vivo and in vitro. Thus, in some embodiments, senescent cells can be identified by screening for such genetic abnormalities and mutations. Thus, for example, another method to determine whether senescence might play a functional role in the pathology of a disease is to assay whether the disease tissue contains greater numbers of cells that are polyploid or have mutations in their mtDNA.

In addition, senescent cells often downregulate genes involved in proliferation and extracellular matrix production, and upregulate inflammatory cytokines and other molecules known to modulate the microenvironment or immune response. Consistent with the role of cellular senescence as a barrier to malignant transformation, senescent cells activate the p53 and p16/Rb tumor suppressor pathways. p53 promotes senescence by transactivating genes that inhibit proliferation, including the p21/Cip1/Waf1 cyclin-dependent kinase inhibitor and miR-34 class of microRNAs. In contrast, p16INK4a promotes senescence by inhibiting cyclin-dependent kinases 2 and 4, thereby preventing Rb phosphorylation and allowing Rb to promote a repressive heterochromatin environment that silences certain proliferation-associated genes. Although the p53 and p16/Rb pathways act in parallel to promote senescence, their relative contribution to the program can be cell type dependent. Thus, another method to determine whether senescence might play a functional role in the pathology of a disease is to assay whether cells in the diseased tissue activate the p53 and/or p16/Rb tumor suppressor pathways.

In another embodiment, a method to determine whether senescence might play a functional role in the pathology of a disease is to assay whether cells in the diseased tissue have a change in the expression level of genes associated with cellular aging. Exemplary biomarkers for this purpose include, but are not limited to, p53, p21, p15, and PAI1. Other markers whose expression increases in senescent HDFs (human diploid fibroblasts) include osteonectin, fibronectin, apolipoprotein J, smooth muscle cells 22 (SM22), and type II (1)-procollagen. Senescent cells also display an increased activity of metalloproteinases, which degrade the extracellular matrix. Senescent cells also have a decreased ability to express heat shock proteins both in vivo and in vitro. In addition, in vitro aging makes HDFs lose c-fos inducibility by serum.

Telomeres are non-coding regions at the tips of chromosomes. In vertebrates, they are composed of repeated sequences of TTAGGG. During in vitro aging, the telomeres shorten gradually in each subcultivation. The same process might occur in vivo too. Thus, methods that assess telomere shortening can also be used to assess the level of senescence in tissues.

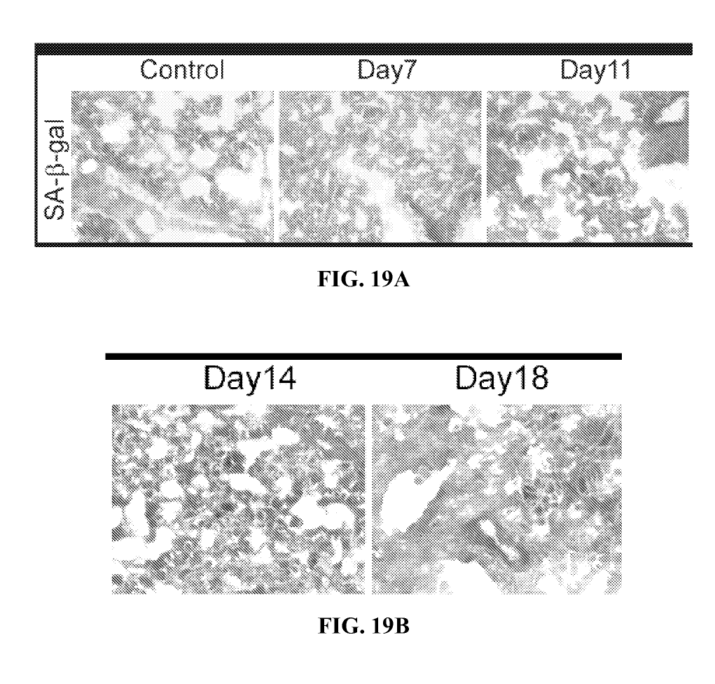

The techniques and approaches described in Example 2 for identifying and assessing senescent cell accumulation in the fibrotic liver is applicable to determining whether other fibrotic tissues contain an accumulation of senescent cells. For example, FIG. 19 shows that senescent cells accumulate in the fibrotic lung tissue as indicated by an increase in SA-β-gal positive staining.

### 4.2 Cellular Senescence Limits Fibrosis in the Liver

Fibrosis arises as part of a wound healing response that maintains organ integrity following catastrophic tissue damage, but can also contribute to a variety of human pathologies, including liver cirrhosis. Here, it is demonstrated that cellular senescence acts to limit the fibrogenic response to tissue damage, thereby establishing a role for the senescence program in pathophysiological settings beyond cancer. The Figures and Examples demonstrate that senescent cells are in fibrotic lung tissue and fibrotic livers of CCl4 treated mice, and that the senescent cells in fibrotic livers arise from activated hepatic stellate cells—a cell type that initially proliferates in response to hepatocyte cell death and is responsible for the extracellular matrix production that is the hallmark of the fibrotic scar.

Liver cirrhosis involves dramatic changes in all cellular components of the liver, being associated with hepatocyte cell death, activation of Kupffer cells and HSCs, and the invasion of inflammatory cells. Previous reports have identified SA-β-gal positive cells in cirrhotic livers and suggested that these cells may arise from damaged hepatocytes. However, as shown herein, the immunotype of senescent cells together with their location along the fibrotic scar indicates that the majority of these arise from senescent activated HSCs. Thus, it is surprising to find that the senescence of activated HSCs limits the accumulation of fibrotic tissue following chronic liver damage, and facilitates the resolution of fibrosis upon withdrawal of the damaging agent. Furthermore, treatments that increase or decrease the number of senescent cells in the liver have an inverse effect on activated HSC accumulation and fibrosis, and livers from mice lacking the key senescence regulators display an aberrant expansion of HSCs and enhanced fibrogenic response. Senescent hepatocytes might also be present in the liver in the later stages of liver disease.

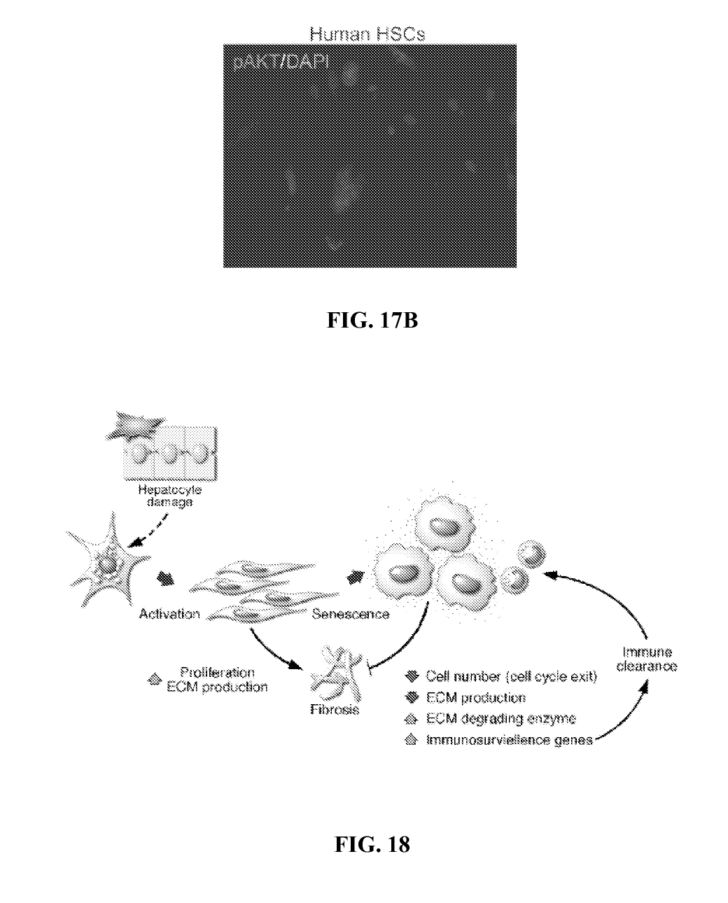

The reason why activated HSCs eventually senesce remains to be determined. While telomere shortening is the driving force of replicative senescence in cultured human cells (Campisi and d'Adda di Fagagna, 2007, also incorporated by reference with respect to telomere-related methods), mouse cells have long telomeres that probably could not shorten sufficiently to trigger senescence during the six week treatment period implemented in the Examples. By contrast, a similar phenomenon of proliferation and senescence has been described in the context of senescence induced by pro-mitogenic oncogenes in both mouse and human cells. In some of these settings, senescence is mediated by hyperactive Akt signaling and, as shown herein, phosphorylated (active) AKT was detected in activated HSCs present in fibrotic mouse livers or that had senesced in culture (FIG. 17). Although correlative, these results are consistent with the possibility that the senescence of activated HSCs results from the hyperproliferative signals that trigger their initial expansion.

It is shown herein that the senescence of activated HSCs provides a barrier that limits liver fibrosis. The hallmark of cellular senescence is its stable cell cycle arrest and this disclosure shows that this process can be triggered acutely in cultured HSCs and is associated with the downregulation of many cell-cycle regulated genes. Undoubtedly, the enforced cell cycle arrest of activated HSCs in vivo provides a brake on the fibrogenic response to damage by limiting the expansion of the cell type responsible for producing the fibrotic scar. Thus, in one embodiment, the invention provides methods for treating fibrosis by increasing senescence in the fibrotic tissue by promoting the cell cycle arrest of myofibroblasts or activated HSCs. The disclosure provides further details below regarding how this can be accomplished.

In addition to halting proliferation, senescent cells—including the activated HSCs studied in the Examples—can also display dramatic changes in their secretory properties. For example, senescent cells downregulate genes encoding extracellular matrix components and upregulate extracellular matrix degrading enzymes (e.g. matrix metalloproteinases), although the biological consequences of these effects have not been considered. In addition, senescent cells typically upregulate a plethora of genes known to stimulate immune surveillance. Without being bound by theory, it is proposed that these changes contribute in a coordinated way to restrain fibrosis—on one hand by limiting the secretion of fibrogenic proteins and degrading those that are present and, on the other, signaling the immune clearance of the expanded population of activated HSCs (FIG. 18). Thus, senescence represents a homeostatic mechanism that enables the tissue to return to its pre-damaged state and is broadly relevant to other wound healing responses. As such, the invention provides methods for treating fibrosis comprising modulating senescence, which includes promoting senescence of cell-types that contribute to the formation of fibrotic scars and/or promoting the killing of senescent cells in the fibrotic tissue.

The mechanism of immune clearance of senescent activated HSCs results from the cytotoxic action of natural killer cells, although other immune components contribute as well. Hence, an antagonist of NK cell function delays the clearance of senescence cells and the resolution of fibrosis, whereas an agent that stimulates the NK cell activation has the opposite effect. Interestingly, a previous report suggested that NK cells might target a fraction of activated HSCs in fibrotic livers (Radaeva et al., 2006), though what signaled this attack was not clear. Although one can not exclude the possibility that spontaneous apoptosis or other modes of cell death contribute to the clearance of activated HSCs in vivo, the present studies indicate that, by activating immune surveillance factors, senescent cells identify themselves to the immune system enabling their efficient clearance—a process that shown herein that can be recapitulated in vitro. Thus, in some embodiments, the invention provides methods for treating fibrosis comprising increasing the killing and/or clearance or removal of senescent cells in fibrotic tissues by administering to the subject an immunostimulatory agent that can increase the numbers of innate immune cells to the fibrotic tissue and/or increase the numbers of activated innate immune cells in the fibrotic tissue.

Although further details to the mechanism are needed, it is shown here that senescent activated HSCs have significantly higher expression of MICA, a ligand of NK cell receptor NKG2D. Of note, Rae family proteins, the NKG2D ligands in mice, are upregulated in response to DNA damage, which also is a trigger for cellular senescence. Thus, in one embodiment, a method for treating fibrosis comprises increasing the killing and/or clearance of senescent cells in the fibrotic tissue by administering to the subject an antibody that binds to MICA. In one embodiment, the antibody is administered directly into the fibrotic tissue. In another embodiment, the antibody is bivalent and comprises a specificity for MICA and another cell surface protein upregulated on the senescent cell as compared to its non-senescent precursor state. In another embodiment, a method for treating fibrosis comprises administering liposomes that are modified to have on its outer surface at least the extracellular domains of NKG2D, such that these liposomes are preferentially targeted to senescent cells that upregulate MICA. These liposomes can contain toxins to kill the senescent cell or expression vectors that can promote senescence as described herein.

Other cell surface proteins that may be upregulated on senescent cells include, but are not limited to, ULBP2, PVR, and CD58. In other embodiments, the antibody binds specifically to at least an antigen on ULBP2, PVR, or CD58. In one embodiment, the antibody must comprise a constant domain capable of being bound by an Fc-receptor on an innate immunity cell in a manner sufficient to mediate cell-killing by the innate immunity cell. In other embodiments, the antibody is conjugated to a toxin/radioactive/chemical moiety such that internalization by the antibody causes cell death.

Previously, it was shown that activation of endogenous p53 in murine liver carcinomas induced senescence and tumor regression in vivo (Xue et al., 2007). Tumor regression was associated with an upregulation of inflammatory cytokines and immune cell adhesion molecules, and several components of the innate immune system contributed to the clearance of senescent cells. The demonstration herein that senescent activated HSCs can be targeted through a similar mechanism further underscores the fact that senescent cells can turn over in vivo to resolve a tissue pathology. Still, not all senescent cells may be targets for the immune system. For example, in the context of benign melanocytic nevi, the accumulation of senescent cells in aged tissues may be related in part to the established decline in immune system function with age. Interestingly, consistent to what is observed in the mouse model studied here, other clinical data suggests that immuno-suppressed patients more rapidly progress to liver cirrhosis, while immuno-stimulatory therapy has a protective effect. The present studies indicate that immuno-stimulatory therapy to enhance senescent cell clearance is a promising treatment of patients with liver fibrosis, especially in its early stages or following short term exposure to hepatotoxic agents. Thus, in some embodiments, a method for treating fibrosis comprises administering to a subject one or more compounds (“compounds” is meant to be used broadly, and includes small molecule compounds, peptides, proteins, etc.) that is capable of causing the activation of resident innate immune system cells in a fibrotic tissue and/or is capable of causing the recruitment (or an increase in recruitment) of innate immune system cells from the periphery to the fibrotic tissue. Further details on such methods are described in subsequent sections).

Without being bound by theory, this model is proposed: Following tissue damage, HSCs (or equivalent cells in non-liver tissues) become activated and proliferate intensely, senesce, and are eventually cleared to protect the liver (or other damaged tissue) from an excessive fibrogenic response to acute injury. However, in response to chronic tissue damage, for example, as produced by viral hepatitis or fatty liver disease, continual rounds of hepatocyte death and activated HSC (myofibroblast) proliferation allow the production of senescent cells to outpace their clearance, contributing to persistent inflammation and advancing fibrosis. Such a state, while initially beneficial, may eventually trigger the aberrant proliferation and transformation of damaged hepatocytes, leading to cancer. In fact, prior mixing experiments indicate that senescent fibroblasts can promote the transformation of premalignant epithelial cells in vivo. Such a model provides one explanation for how cirrhosis predisposes to hepatocellular carcinogenesis and may be relevant to other settings where fibrosis occurs.

### 4.3 Methods for Treating or Limiting Fibrosis

The therapeutic methods of the invention are applicable to any fibrotic tissue, including liver, lung, atherosclerotic tissue, skin, pancreas, or prostate. For any target tissue, the methods can comprise increasing the number of senescent cells in the fibrotic tissue and/or increasing the killing and/or clearance of senescent cells in the fibrotic tissue. In some embodiments, the methods comprise both steps of increasing the number of senescent cells in the fibrotic tissue and/or increasing the killing and/or clearance of senescent cells in the fibrotic tissue.

The methods are not meant to be limited to removing only senescent cells that were previously myofibroblasts or other activated cell-types that were producing extracellular matrix or other components of the fibrotic scar. Rather, the removal of senescent cells in general in the fibrotic tissue is preferred because an overabundance of senescent cells can disrupt normal tissue microenvironments and architecture and promote tumorigenesis.

Further, because it is shown herein that the senescence machinery limits fibrosis, the methods can comprise at least the step of increasing or promoting the senescence of cells that contribute to the formation of fibrotic scars, such as myofibroblasts or other extracellular matrix producing cells. In some embodiments, methods that increase the senescence of cells also have the step of increasing the removal of senescent cells to avoid accumulation, such that the overall effect is a more robust senescence machinery or cycle that will lead to faster or more efficient fibrosis resolution.

In one embodiment, methods for treating fibrosis comprises promoting senescence by activating p53 or by transactivating genes that inhibit proliferation, including the p21/Cip1/Waf1 cyclin-dependent kinase inhibitor and miR-34 class of microRNAs. In one embodiment, promoting senescence in fibrotic tissue comprises administering replication deficient retrovirus particles or expression vectors (including but not limited to expression vectors based on alpha virus, adeno-associated virus, and adenovirus) that comprise p53 coding sequence, p21/Cip1/Waf1 cyclin-dependent kinase inhibitor, or a miR-34 microRNA. In one embodiment for treating liver fibrosis, expression vectors (which includes viral vectors) comprise a p53 coding sequence under control of the GFAP promoter, which is HSC specific. These can be administered directly to the fibrotic tissue. In another embodiment, promoting senescence comprises promoting p16INK4a, inhibiting cyclin-dependent kinases 2 and 4, preventing Rb phosphorylation, and/or allowing Rb to promote a repressive heterochromatin environment that silences certain proliferation-associated genes. In one embodiment, promoting senescence in fibrotic tissue comprises administering replication deficient retrovirus particles or expression vectors that comprise a p16INK4a coding sequence. In one embodiment, promoting senescence in fibrotic tissue comprises administering replication deficient retrovirus particles that comprise a sequence coding for dsRNA or short-hairpin RNA molecule that can cause post-transcriptional silencing of cyclin-dependent kinases 2 and/or 4 via RNA interference.

In any embodiment of the invention that relates to the delivery of siRNA or shRNA molecules, such molecules can be delivered, for example, to a subject through the use of nonintegrating or integrating viruses. Nonintegrating viruses include adenovirus, adeno-associated virus, or herpes simplex virus. Nonintegrating viruses can mediate stable expression of the siRNA or shRNA molecule in nondividing cells. Integrating viral vectors are appropriate if persistent knockdown (stable suppression) is desired. Murine retrovirus-based vectors are an exemplary integrating vector, as these viruses are amphotropic and can infect both murine and human cells. Other integrating vectors include lentiviruses, such as HIV, FIV, and EIAV based vectors.

In another embodiment, a method for increasing senescence comprises administering liposomes that can preferentially target activated HSC cells. For example, liposomes can be modified such that their outer surface can comprise ligands to cell surface proteins present or upregulated on activated HSCs, and such liposomes can contain toxins, expression vectors that express genes or RNA molecules that can promote senescence, or low dose DNA damaging agents (the direct delivery method was recently described in Adrian et al, J. of Liposome Research, 2007, 17; 205-218, which is hereby incorporated by reference).

Methods for treating fibrosis in the liver can be with respect to essentially any type of liver disease or injury that involves the formation of fibrotic tissue. For example, the liver disease or injury can comprise, for example, chronic HCV infection, liver injury due to alcohol, age, obesity, diabetes, hypertriglyceridemia, autoimmune hepatitis, alcoholic hepatitis, and toxins.

In some embodiments, the methods for treating fibrosis in the liver is focused on intermediate to advanced fibrosis (cirrhosis). Without being bound by theory, in cases where the degree of fibrosis is intermediate to advanced, the rationale is to eliminate the ongoing accumulation of senescent cells as killing and clearing this accumulation along with elimination of primary cause of the disease if possible will help to improve liver function, resolve fibrosis or at least stop its further development and prevent potential progression from fibrosis to tumorigenesis.

In some embodiments, the methods for treating fibrosis in the liver is focused on low levels of fibrosis. Without being bound by theory, in cases where the degree of fibrosis is minimal to intermediate, a strategy to specifically target senescent as opposed to activated HSCs may be preferred because for acute injury, activated HSCs are a fundamental part of the healing process. However, even for chronic fibrosis, a strategy to specifically target senescent cells for killing as opposed to activated HSCs may be preferred because activated HSCs help not only to repair damaged tissue but they are also involved in promoting the proliferation of new hepatocytes.

The degree of fibrosis can be determined in a subject by various methods. Histologic examination of liver biopsy tissues is a standard method for assessing the degree of fibrosis, and standard grading scores are used such as Metavir (stages I-IV) and Ishak score (stages I-V). Staining of extracellular matrix proteins by Sirius red can be used to quantify the degree of fibrosis. Serum levels of proteins such as N-terminal propeptide of type III collagen, hyaluronic acid, tissue inhibitor of metalloproteinase type I (TIMP-1), and YKL-40 can be also be used. Ultrasonography, computed tomography, and MRI can also be used.

In various embodiments, the methods for treating fibrosis comprise the step of increasing the killing/clearance/removal of senescent cells in the fibrotic tissue. This can be accomplished by general and/or specific approaches. A general approach is to administer to the subject an immunostimulatory compound that results in an increase in the numbers of activated innate immunity cells in the fibrotic tissue and/or an increase in the recruitment of innate immunity cells to the fibrotic tissue.

Innate immune system cells include but are not limited to mast cells, phagocytes (such as macrophages, neutrophils, and dendritic cells), basophils, eosinophils, natural killer cells, natural killer T-cells, and gamma-delta T-cells. In one embodiment, increasing the killing/removal of senescent cells in fibrotic tissue comprises increasing the number of activated NK cells in the fibrotic tissue and/or increasing the recruitment of NK cells to the fibrotic tissue from the periphery or other compartments including the bone marrow.

Immunostimulatory compounds that can be used to generally stimulate the innate immune system include, but are not limited to, IFN-α, IFN-γ, IL-1, IL-2, IL-6, IL-8, IL-13, IL-15, IL-18, IL-24, BMP2, GDF15, CXCL1, CXCL2, CXCL3, CXCL5, CXCL12, CCL20, CCL15, CCL26, LIF, CNTF, BSF3, and CTF1. As used herein, “stimulate” includes activation and/or recruitment of innate immune system cells in/to the fibrotic tissue. One or more of these compounds may be administered to the subject in an amount sufficient to increase the numbers of activated innate immune cells in the fibrotic tissue and/or in an amount sufficient to increase the numbers of innate immune cells in the fibrotic tissue (i.e., increase the recruitment or migration of such cells from the periphery or other compartments to the fibrotic tissue).

Immunostimulatory compounds that can be used to preferentially stimulate NK cells include, but are not limited to, agonists of NKp30, NKp44, NKp46, NKG2D receptors; and agonists of SLAM-related receptors (SRR) including agonists of 2B4 (CD244), NTB-A, CS1 (CRACC). In one embodiment, one or more of such agonists are administered to the subject, either systemically or directly to the fibrotic tissue. As used herein, agonists include but are not limited to small molecules, peptides, proteins, antibodies, fusion proteins.

In one embodiment, IL-15 alone or in combination with IL-18 are used to increase the recruitment of innate immune system cells to the fibrotic tissue by administering the cytokine(s) directly to the tissue.

In another embodiment, natural killer (NK) cells can be isolated from the subject, expanded and/or activated in culture, and administered to the subject, either directly to the fibrotic tissue or intravenously. Peripheral blood can be isolated from the subject and the NK cell fraction can be sub-isolated by magnetic beads or flow cytometry by focusing on NK1.1⁺ CD3⁻ cells.

A specific approach for increasing the killing/clearance/removal of senescent cells in the fibrotic tissue can comprise administering to the subject a compound that preferentially causes the killing/removal of senescent cells in the fibrotic tissue. This can be accomplished, for example, by administering an antibody or combination of antibodies that target one or more upregulated or overexpressed cell surface molecules on a senescent cell in the fibrotic tissue (upregulated or overexpressed with respect to the same cell-type prior to its senescent state). In a preferred embodiment, the upregulated cell surface molecule(s) are with respect to senescent cells in the fibrotic tissue that were contributing to the formation of fibrotic scars in the tissue—which can include or be exemplified for example by cells that are producing extracellular matrix components that form the fibrotic scar. Exemplary upregulated cell surface markers that can be used to target senescent cells include, but are not limited to, ligands of NK activation receptors (including ligands of NKp30, NKp44, NKp46, NKG2D receptors such as MICA, a ligand of NK cell receptor NKG2D), ULBP2, PVR, and CD58. In one embodiment, the antibody is multivalent and binds to at least two upregulated cell surface proteins. In one embodiment, the antibody must comprise a constant domain capable of being bound by an Fc-receptor on an innate immunity cell in a manner sufficient to mediate cell-killing by the innate immunity cell. In other embodiments, the antibody is conjugated to a toxin/radioactive/chemical moiety such that internalization by the antibody causes cell death. In another embodiment, liposomes can be coated with ligands that bind to cell-surface proteins that are upregulated on senescent cells, such that the liposomes preferentially deliver toxins or genes that can promote the killing or apoptosis of senescent cells.

### 4.4 Methods of Screening for Potential Therapeutic Compounds for Treating Fibrosis

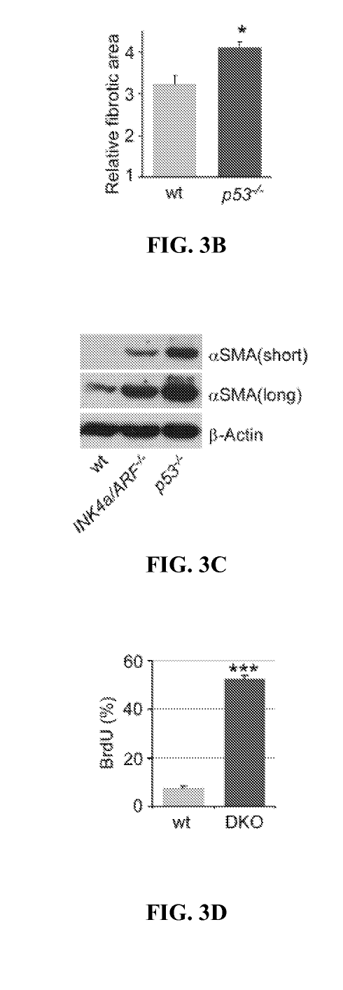

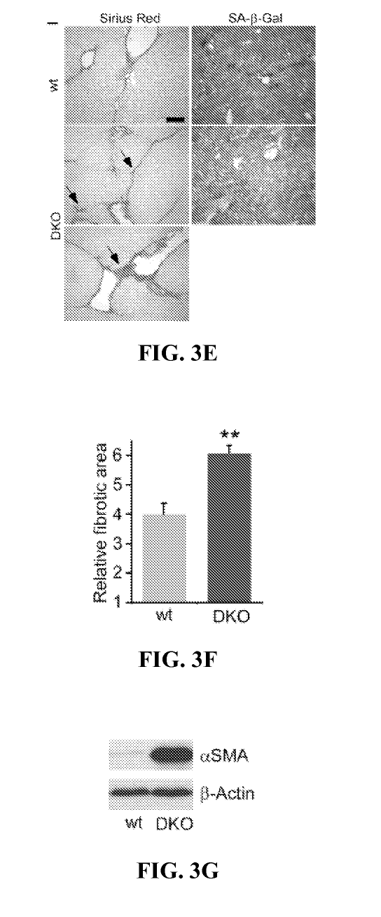

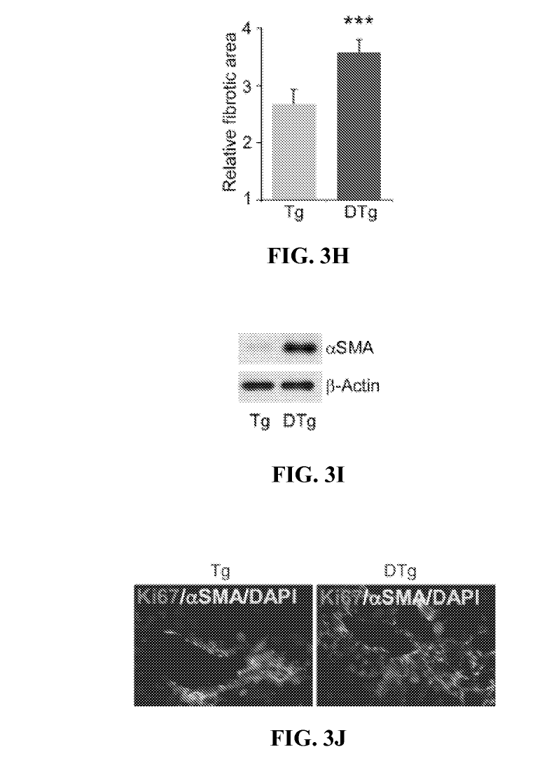

Mouse HSC could be extracted from mouse livers (they can senesce in vitro—FIG. 3D) and then growing/senescent cells are co-incubated with mouse NK cells or macrophages or NKT cells or any other immune cells and any compound could be tested in this system.

In other embodiment, the procedures used in Example 7 can be adapted for methods of screening compounds to identify potential candidates for use as therapeutic drugs for fibrosis-related disorders and diseases.

For example, senescent IMR-90 cells (senescence induced by etoposide, replicative exhaustion, or oncogenic ras, for example) can be co-cultured with an innate immune system cell line, such as YT (NK cell line). For example, a test compound, whether a small-molecule, an antibody, fusion protein, etc., can be added to the co-culture to assess whether its addition causes an increase in the preferential association between senescent cells and NK cells and/or whether its addition causes an increase in the specific killing of the senescent cell.

In one embodiment, screening methods can be based on the difference between senescent and growing cells with respect to their sensitivity to NK cells. For example, IMR-90 cells growing in culture are not attacked by YT cells (NK cell line) and remain attached to the culture dish. By contrast, senescent IMR-90 cells readily attract YT cells, and undergo apoptosis and detach from the surface of the dish. Thus, a test compound can be added to a mixed culture of growing and senescent IMR-90 cells (or other type of myofibroblast cell line) and NK cells (or other type of innate immune cell) to see whether the addition of the test can cause a specific increase in the apoptosis and detachment of myofibroblasts that is NK cell dependent. If the addition of the test compound causes the killing of the growing cells (or the NK cells), then the test compound is not considered to promote specific innate immune system cell-mediated killing of the senescent cell. In one aspect, the identification of test compounds that cause a specific increase in the apoptosis and detachment of senescent myofibroblasts is desired. In another aspect, the identification of test compounds that cause a specific increase in the apoptosis and detachment of senescent myofibroblasts is desired, where this effect is NK-cell dependent.

In another embodiment, test compounds can be screened to assess whether they can cause an increase in cytotoxic activity towards senescent cells. Such an assay can be assessed by a quantitative in vitro cytotoxicity assay. For example, crystal violet staining of cell populations at various time points can be used to show whether there is an increase in cytotoxic activity caused by the addition of a test compound.

Compounds that can promote the increase in the specific killing of senescent cells can be further tested using the in vivo models of Examples.

### 5. EXAMPLES OF THE INVENTION

The following Examples are not meant to limit the invention. The Examples provide exemplary teachings and can be modified or varied to the different embodiments of the invention as understood by one of skill in the art.

### Example 1

### Experimental Procedures

The following experimental procedures were used in the Examples.

Animals. Genotyping protocols of p53^{-/-}, INK4a/Arf^{-/-} and TRE-shp53 mice were previously described and are incorporated by reference (Dickins et al., 2007, Nat. Genet., 39, 914-921; Schmitt et al., 2002, Cell, 109, 335-346). GFAP-tTA mice were obtained from the Jackson Laboratory. Wild type, p53^{-/-}, INK4a/Arf^{-/-} and p53^{-/-};INK4a/Arf^{-/-} mice were treated twice a week with 12 consecutive i.p. (intraperitoneal) injections of 1 ml/kg CCl4 to induce liver fibrosis. GFAP-tTA;TRE-shp53 mice were treated similarly for 2 weeks. Animals were sacrificed 48-72 hours after the last injection and their livers used for further analysis. To modify NK cell function, mice were treated three times weekly either i.v. with an anti-Asialo-GM1 antibody (25 μl in 200 μl saline, Wako, Va., USA) for 10 or 20 days or i.p. with polyI:C (Sigma, USA) 1 mg/kg.

Histological analysis. Paraffin embedded tissue sections were stained with hematoxylin-eosin for routine examination, or with Sirius Red for visualization of fibrotic deposition. At least 3 whole sections from each animal were scanned by Laser Scanner Cytometry (CompuCyte, MA) for fibrosis quantification. These images were quantified using NIH ImageJ software (http://rsb.info.nih.gov/ij/). We calculated the amount of fibrotic tissue in diseased animals relatively to the basal amount of Sirius Red staining present in normal liver.

Detection of SA-β-gal activity was performed as described previously which is hereby incorporated by reference (Serrano et al., 1997, Cell, 88, 593-602) at pH=5.5 for mouse tissue and pH=6.0 for human cells. Frozen sections of liver tissue, or adherent cells were fixed with 0.5% Gluteraldehyde in PBS for 15 min, washed with PBS supplemented with 1 mM MgCl2 and stained for 5-6 hrs in PBS containing 1 mM MgCl2, 1 mg/ml X-Gal and 5 mM of each Potassium ferricyanide and Potassium ferrocyanide. Sections were counterstained with Eosin.

Immunostaining was performed as previously described which is hereby incorporated by reference (Xue et al., 2007). The following antibodies were used: Ki67 (Dianova, Germany), p21 (BD Pharmingen, USA), αSMA (DakoCytomation, Denmark), p16, p53, Desmin and GFAP (all from Santa Cruz, USA). Anti-HMGA1 antibodies were raised in rabbits immunized with peptide corresponding to amino acids 79 to 94 in HMGA1 protein and found to be reactive with HMGA1 (and not cross-reactive with HMGA2) (Narita et al., 2006, Cell, 126, 503-514, which is hereby incorporated by reference). AlexaFluor conjugated secondary antibodies were used for signal detection.

Electron microscopy. Samples of mouse liver were fixed, dehydrated and embedded in Epon-Araldite (Electron Microscopy Sciences, Pa., USA). Sections were contrasted and imaged in a Hitachi H7000T transmission electron microscope.

Tissue culture. Human IMR-90 foetal lung fibroblasts (ATCC) and primary human hepatic myofibroblasts (activated HSCs) (Dominion Pharmakine, Spain) were grown in standard conditions (Narita et al., 2003). Senescence was induced by prolonged culturing, etoposide (100 μM, Sigma, USA) treatment, or infection of IMR-90 cells with oncogenic rasV12 as described (Narita et al., 2003). For in vitro cytotoxicity assays, growing or senescent cells were plated in 6-well plates at 50,000 cells per well. 5×10⁵ YT cells (from DSMZ, Germany) were subsequently added to target cells. The plates were incubated under normal conditions for 12 hours, and then NK cell cytotoxicity was determined using crystal violet staining of remaining adherent cells or followed with a Zeiss AxioObserver microscope equipped with 37° C. incubator hood and 6.3% CO₂ cover.

Immunoblotting. Liver tissue was lysed in Laemmli buffer using a tissue homogenizer. Equal amounts of protein were separated on 12% SDS-polyacrylamide gels and transferred to PVDF membranes. Detection was performed using anti-αSMA (DakoCytomation, Denmark), anti-13Actin (AC-15, Sigma, USA).

Expression array analysis and quantitative RT-PCR. RNA preparation, cDNA synthesis and quantitative PCR were performed as described previously which is hereby incorporated by reference (Xue et al., 2007). Affymetrix Human Genome U133 Plus 2.0Array were used to identify genes expressed in HSC. Gene Ontology (GO) (http://www.geneontology.org/) and KEGG pathway (http://www.genome.jp/kegg/pathway.html) analysis was performed on up-regulated and down-regulated genes using g: Profiler web tool (http://biit.cs.ut.ee/gprofiler/).

HSC isolation was performed as described in Zhang et al, World J. Gastroenterol. 2006; 12(12): 1918-1923 with slight modifications, which is hereby incorporated by reference. Cells were cultured for 3 weeks prior to staining.

Detection of microRNA expressed from TRE-shp53 transgene was performed using Taqman MicroRNA Assay kit with custom designed specific primers (Applied Biosystems).

Immunohistochemistry on formalin-fixed, paraffin-embedded human liver tissues was performed using anti-p16 (Abcam), anti-p21, clone EA10 (Oncogene Sciences) and anti-smooth muscle actin (SMA), clone 1A4 (Dako). In brief, sections were deparaffinized, rehydrated, and epitope retrieval was performed. Endogenous peroxidase activity was blocked with hydrogen peroxide. Primary antibodies were detected by the application of a biotinylated goat anti-mouse or goat anti-rabbit, followed by the application of streptavidin-horseradish-peroxidase conjugate. The complex was visualized with 3,3 diaminobenzidene and enhanced with copper sulfate. Slide where counterstained with hematoxylin. Appropriate positive and negative controls were included.

For live cell imaging, growing and senescent IMR-90 cells were plated at 5×10⁴ in 6-well plates (PO6G-1.5-20F, MatTek, Mass., USA) pre-coated with 0.1% gelatin. Cells were incubated for 12 hours at standard conditions before adding the YT cells at 5×105 cells in RPMI containing 10% FCS and antibiotics. Cells were observed with a Zeiss AxioObserver microscope with ×20 objective equipped with 37° C. incubator hood and 6.3% CO2 cover following YT cell addition. DIC images from 10 independent positions per well were collected simultaneously every 5 min for 12 hours with a Zeiss AxioCam and the images processed for time-lapse movies using AxioVision 4.6 software.

### Example 2

### Senescent Activated Stellate Cells Accumulate in the Cirrhotic Liver

The following teachings can be adapted to determine whether senescent cells accumulate in other fibrotic tissues besides liver. For example, after treatment to a model organism to cause damage/fibrosis in a target tissue, the tissue can be analyzed for senescent cell accumulation as described below. Fibrotic tissues that are identified to accumulate senescence cells can be treated by the methods described supra.

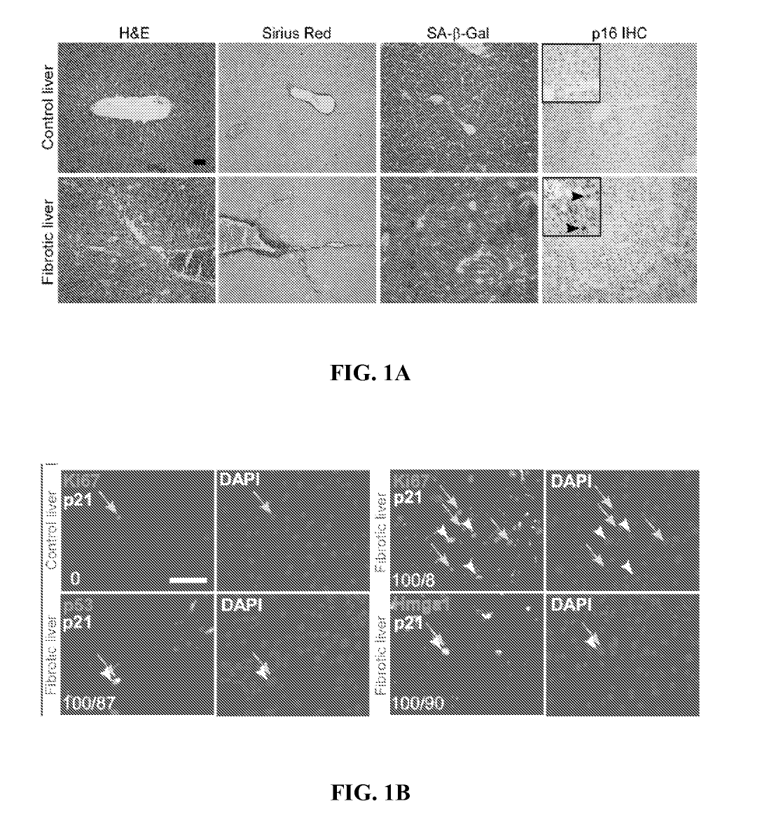

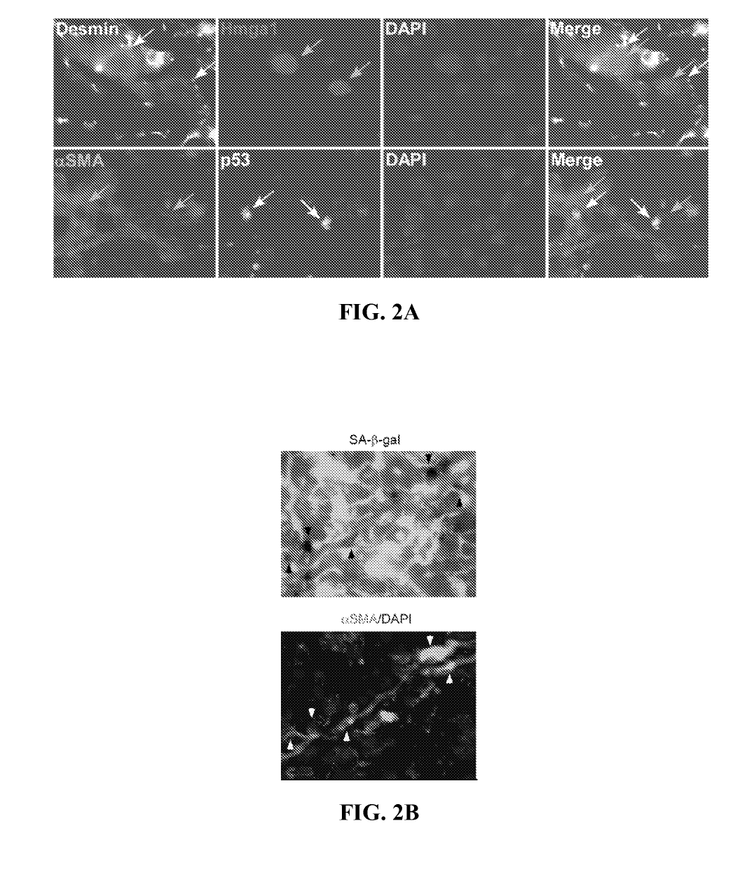

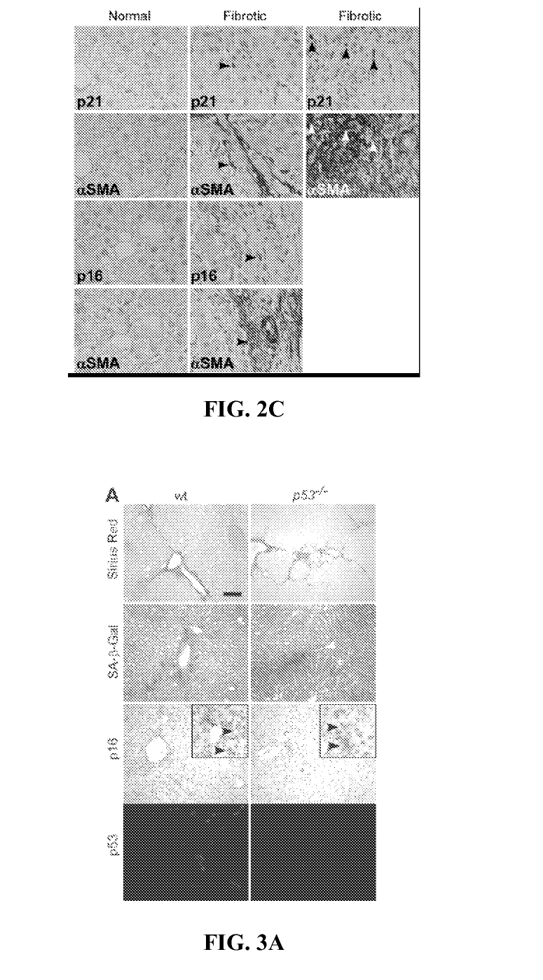

To investigate the relationship between fibrosis and cellular senescence, 7-9 week old female mice were subjected to a six week treatment with CCl4, a chemical widely used to induce fibrosis in experimental animals (Bataller and Brenner, 2005, J. Clin. Invest. 115, 209-218, the contents of which are hereby incorporated by reference). This protocol produced fibrosis as assessed by staining with Hematoxylin-Eosin and Sirius Red, which directly marks the extracellular matrix deposited by activated HSCs (FIG. 1A). Approximately 2% of the liver was Sirius Red-positive as assessed by quantitative laser scanning cytometry, representing a 3 to 4-fold increase over untreated controls. Furthermore, CCl4 treatment produced a dramatic expansion of activated HSCs, which were visualized by immunofluorescence staining of liver sections for the activated HSC markers desmin and α-smooth muscle actin (αSMA) (data not shown, see also FIG. 2).

To identify senescent cells in situ, liver sections from CCl4 and vehicle-treated (control) mice were stained for a panel of senescence-associated markers, including SA-β-gal and proteins such as p16, p21, p53 and Hmga1, which have been causally linked to the senescence program (Collado et al., 2007, Cell, 130, 223-233; Narita et al., 2006, Cell, 126, 503-514; Serrano et al., 1997; the contents of which are hereby incorporated by reference). Cells staining positive for SA-β-gal and each senescence-associated protein accumulated in fibrotic livers, and were invariably located along the fibrotic scar (FIG. 1). These cells typically expressed multiple senescence markers and were not proliferating (only cells with nuclear staining for p21, p53 and Hmga1 were considered positive). For example, of the p21 positive cells identified in fibrotic livers, 87% were positive for p53 immunostaining and 90% were positive for Hmga1 staining (FIG. 1B), whereas only 8% co-expressed the proliferation-association marker Ki-67 despite a general increase in the frequency of Ki-67 positive cells (FIG. 1B). Of note, these senescence markers were not expressed in control livers (FIG. 1B, data not shown). Moreover, senescent cells also accumulated in livers derived from mice treated with DDC (3,5-diethoxycarbonyl-1,4-dihydrocollidine), another agent that produces liver fibrosis and cirrhosis (data not shown).

Although hepatocytes represent the most abundant cell type in the liver, the location of senescent cells along the fibrotic scar in both human (Wiemann et al., 2002), and mouse (FIG. 1B) livers raised the possibility that these cells were derived from activated HSCs, which initially proliferate following liver damage and are responsible for much of the extracellular matrix production in fibrosis. Accordingly, in mouse fibrotic liver sections, the cells that stained positive for the senescence-associated markers p53 and Hmga1 were also positive for the HSC markers desmin and αSMA and, in serial sections, most SA-β-gal positive cells also expressed αSMA (FIG. 2B). Similarly, in serial sections obtained from human cirrhotic livers, cells expressing the senescence markers p21 and p16 co-localized with those expressing αSMA (FIG. 2C). Therefore, senescent activated HSCs accumulate in fibrotic livers.

### Example 3

### Fibrosis Progression is Restricted by an Intact Senescence Machinery

Hepatic stellate cells initially proliferate in response to liver damage, and so it was not obvious how their senescence would ultimately influence the progression of fibrosis. Since p53 contributes to cellular senescence in most murine tissues (Collado et al., 2007), cells derived from mice lacking p53 often show an enhanced proliferative capacity in culture (Sherr, 1998, Genes Dev., 12, 2984-2991). To evaluate the biological impact of senescence on liver fibrosis, the histopathology of livers obtained from wild-type and p53^{-/-} mice treated with CCl4 was initially compared. After six weeks, livers were examined for fibrosis using Sirius Red staining and expression of Tgfb1, a major cytokine upregulated during fibrosis progression (Bataller and Brenner, 2005).

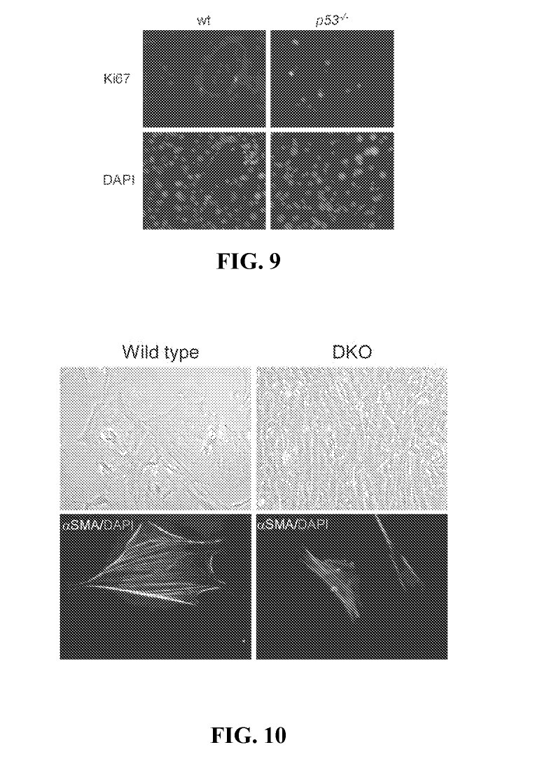

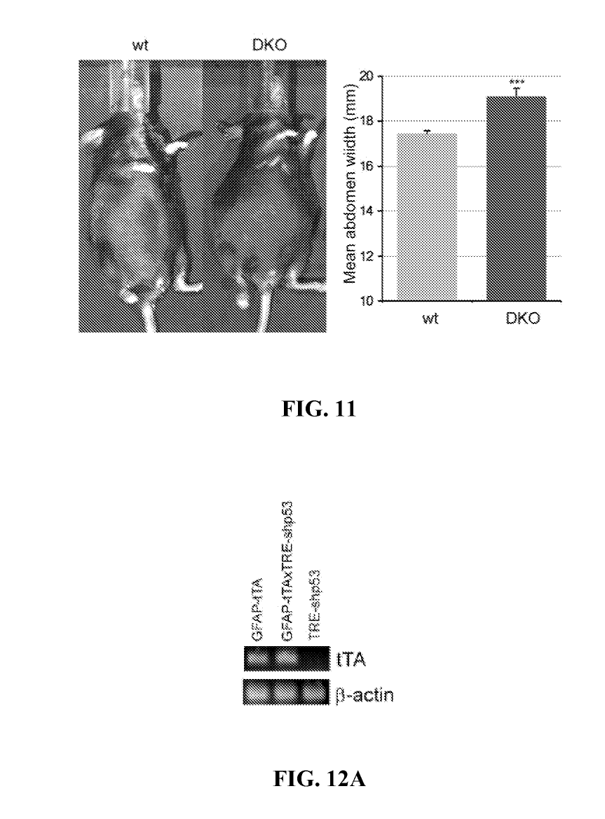

Surprisingly, livers derived from p53^{-/-} mice contained significantly more fibrotic tissue relative to wild type controls (FIGS. 3A,B, and data not shown, p=0.008) and also displayed an increase in Tgfb1 expression (FIG. 8). This increase in fibrosis was associated with an aberrant expansion of activated HSCs as assessed by αSMA expression as a surrogate marker for the abundance of this cell type (FIG. 3C, FIG. 8). Conversely, livers derived from p53^{-/-} mice treated with CCl4 showed more proliferating cells (FIG. 9) and a decrease in SA-β-gal staining compared to wild type controls (FIG. 3A). These observations indicate that, in the absence of p53, liver damage produces fewer senescent cells, and a corresponding increase in activated HSCs, extracellular matrix deposition, and fibrosis.

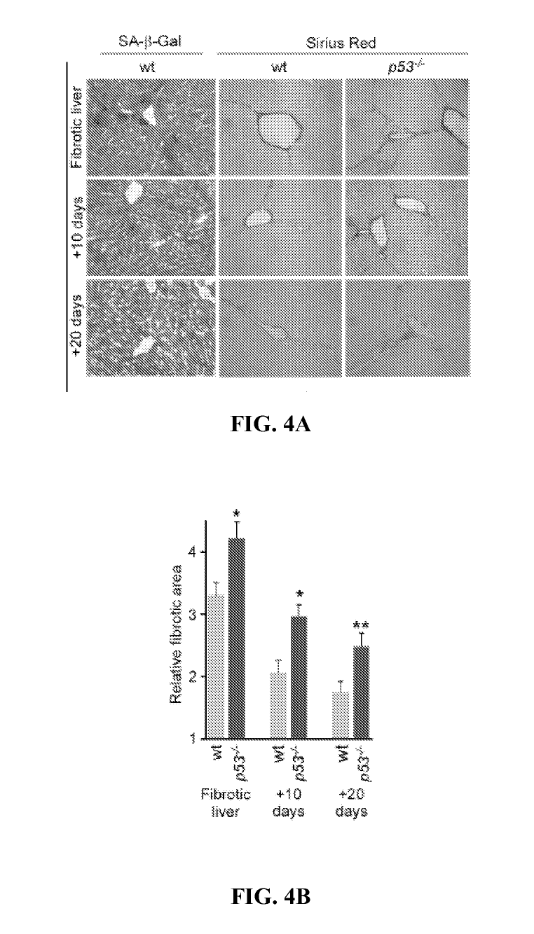

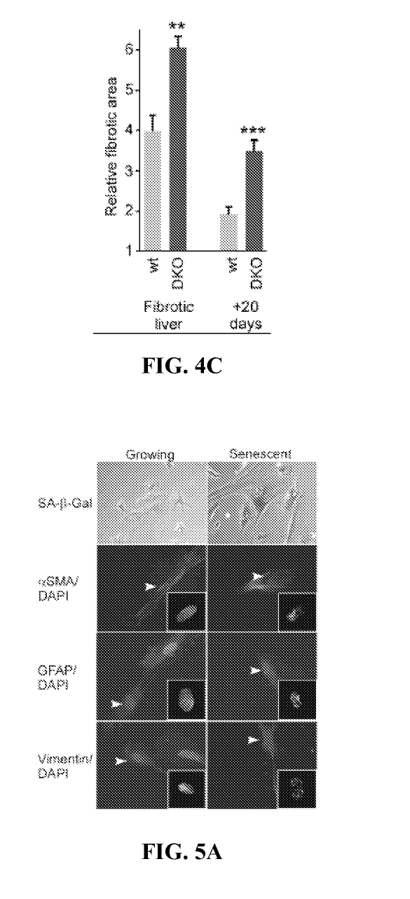

In many cell types, both the p53 and the p16/Rb pathways contribute to senescence such that cells lacking either pathway alone retain a residual senescence response (Serrano et al., 1997). In fact, livers derived from p53^{-/-} mice treated with CCl4 still showed some increase in SA-β-gal positive cells and retained their ability to upregulate p16 (FIG. 3A). Moreover, CCl4 treated livers from INK4a/ARF^{-/-} mice also showed only a partial reduction in senescence [corresponding to an increase in HSCs (FIG. 3C) and fibrosis (data not shown)], and still upregulated p53 (data not shown). To determine the impact of disrupting both loci on senescence and fibrosis in the liver, double knock-out mice were produced, p53^{-/-};INK4a/ARF^{-/-} compound mutant mice. Since less then 5% of the female double mutant mice reached adulthood, only male animals were used in these experiments. Of note, male mice develop more severe fibrosis than females [compare FIG. 4B to 4C], making comparisons within the same sex essential.

Consistent with the predicted consequences of p53 and INK4a/ARF inactivation on senescence, isolated HSCs from double knockout livers did not senesce in culture, showing much less SA-β-gal activity and much more BrdU incorporation compared to wild type cells, which senesced after a few passages (FIG. 3D; FIG. 10; data not shown). Livers derived from CCl4 mice lacking both p53 and INK4a/ARF developed severe fibrosis when compared to wild-type animals, showing a greater than 50% increase in fibrotic area, wider fibrotic scars, and substantially more scar branching (FIGS. 3E, 3F). Moreover, double mutant livers contained few SA-β-gal positive cells (FIG. 3E) and harbored a large increase in activated HSCs as determined by αSMA protein (FIG. 3G) and mRNA (data not shown, 35-fold relative to controls, p=0.02) expression. Double knockout animals also developed clearly visible ascites, one of the clinical manifestations of cirrhosis, resulting in significantly wider abdomens compared to controls (Supplementary FIG. 4, p=0.006). Therefore, activated HSCs lacking both the p53 and INK4a/ARF genes (and thus the ARF/p53 and p16/Rb pathways) fail to senesce and inappropriately expand in response to chronic liver damage, leading to more extracellular matrix production and fibrosis.

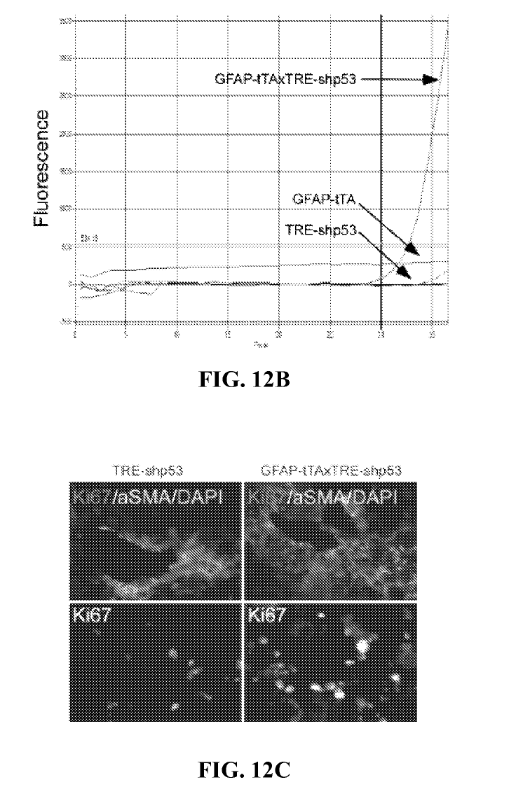

To confirm that the above phenotypes were a result of impaired senescence in activated HSCs and not other liver cell types, p53 expression in HSCs was specifically suppressed and the extent of liver fibrosis and activated HSC proliferation following CCL4 treatment was examined. Transgenic mice harboring a tetracycline response element (TRE) driven short haipin RNA (shRNA) capable of efficiently suppressing p53 expression (Dickins et al., 2007) were crossed to mice harboring a tTA (tetracycline-controlled transactivator) transgene expressed from the GFAP promoter (Wang et al., 2004, Mol. Cell. Neurosci., 27, 489-496, which is hereby incorporated by reference) which, in the liver, is HSC specific. As the tTA transactivaor binds the TRE promoter in the absence of tetracycline, double transgenic mice (DTg) should constitutively express the p53 shRNA, which proved to be the case (FIG. 12). Consistent with observations in p53 null animals, double transgenic mice where p53 was suppressed specifically in HSCs developed significantly more fibrosis than controls (FIG. 3H, p=0.0009); moreover, immunofluorescence studies revealed that their livers contained more proliferating HSCs (Ki-67 and αSMA-positive) (FIGS. 3I, 3J). These data indicate that the senescence of activated stellate cells limits fibrotic progression.

### Example 4

### Cellular Senescence Facilitates the Reversion of Fibrosis

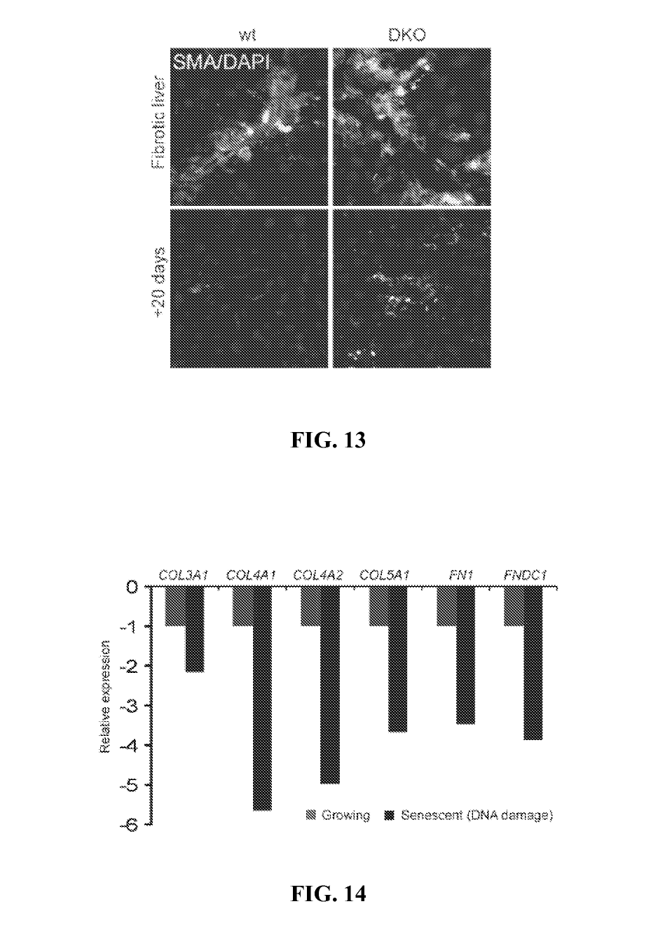

Although the architectural changes that accompany cirrhosis are considered irreversible, it is now evident that fibrosis in patients, even in more advanced stages, can regress following eradication of the disease trigger (Bataller and Brenner, 2005). Accordingly, liver fibrosis in wild type animals resolved within 10 days after stopping CCl4 treatment and was almost undetectable by 20 days (FIG. 4A). The frequency of senescent cells in wild-type livers declined with the reversion of fibrosis, as did the number of HSCs, such that no SA-β-gal positive cells were detected in 20 day post-treatment livers and the amount of αSMA present dramatically declined. In marked contrast, activated HSCs were clearly retained in livers from p53^{-/-} mice at 20 days post-treatment, and this correlated with an impairment in fibrotic reversion (FIGS. 4A,B, p=0.014, p=0.006, 10 or 20 days after the treatment respectively). Even more fibrotic lesions and activated HSCs were retained in p53^{-/-};INK4a/ARF^{-/-} mice at this time point (FIG. 4C, FIG. 13).

Consistent with the impaired clearance of fibrotic tissue in the absence of p53, livers derived from p53^{-/-} animals displayed much higher levels of TGFβ and αSMA following CCl4 withdrawal compared to controls, implying that they maintained greater fibrogenic signaling and more activated HSCs (FIG. 8). p53^{-/-} livers also retained more proliferating (Ki67-positive) cells than wild-type controls (FIG. 9), suggesting p53-deficient activated HSCs can bypass the senescence response, continue to proliferate and deposit extracellular matrix in the scars. Thus, senescence limits proliferation of activated HSCs and facilitates their clearance from the liver.

The teachings in Example 4 can also be applied to determine whether the senescence programs serves to limit fibrosis in other tissues. For example, the mouse models described above, such as the various knock-out and transgenic mice can be used in similar fashion to focus on other target tissues.

### Example 5

### Senescent Activated HSCs Upregulate the Expression of Immune Modulators

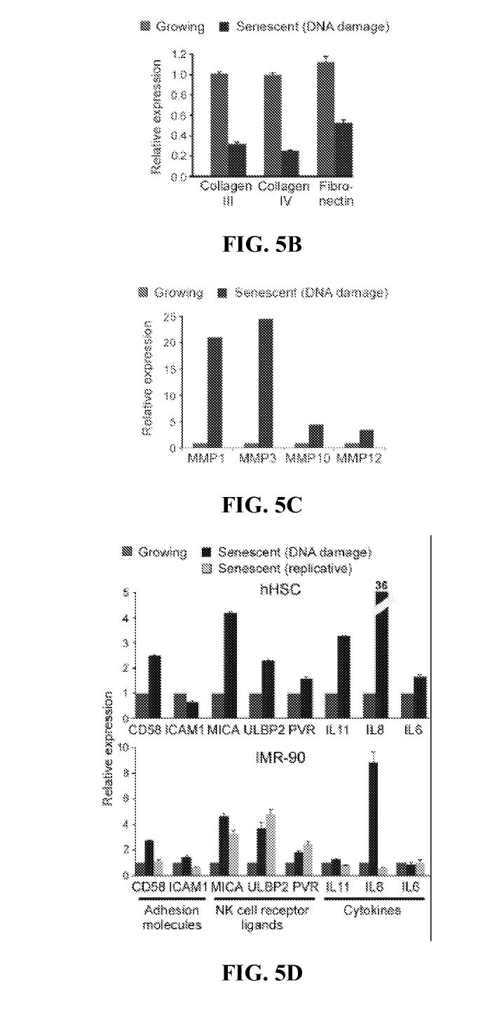

As a first step towards defining how activated stellate cells undergo senescence and are cleared from tissue, the transcriptional profiles of cultured primary human activated HSCs that were proliferating or triggered to senesce by treatment with a DNA damaging agent, etoposide, were compared. Like IMR-90 normal diploid fibroblasts, a cell type in which senescence has been studied extensively, activated HSCs stopped proliferating, accumulated SA-β-gal activity, and acquired senescence-associated heterochromatic foci within several days of etoposide treatment, yet retained the activated HSC markers αSMA, GFAP and Vimentin (FIG. 5A). Thus, by several criteria, etoposide-treated activated HSCs undergo senescence.

Gene expression profiling of two different activated HSC preparations was performed using Affymetrix Human Genome U133 Plus 2.0 Arrays, and the differentially expressed genes analyzed using Gene Ontology (GO) to identify biological processes and pathways that were altered in an unbiased way. Consistent with the proliferative arrest that accompanies senescence, the most significantly overrepresented “Biological Process” term among downregulated genes was “cell cycle” (see Table 1 below, p=1.72E-21), and included genes necessary for cell cycle progression such as CDKN3, CyclinB (CCNB1, CCNB2), CDC20, and the E2F target genes CDC2 (CDC2), CyclinA2 (CCNA2) and Thymidine kinase (TK1). Genes encoding extracellular matrix components were also significantly overrepresented among downregulated genes, including those linked to “extracellular matrix” (Cellular Component term) and “extracellular matrix structural constituent” (Molecular Function term) (p=3.44E-6 and p=1.75E-6, respectively). Interestingly, Collagens type I, III, IV and Fibronectin are constituents of the fibrotic scar (Bataller and Brenner, 2005), and most of these genes were downregulated on the microarray (FIG. 14) and quantitative RT-PCR analyses (FIG. 5B). These observations indicate that the senescence program limits both the proliferative and fibrogenic potential of activated HSCs.

TABLE 1
GO terms and KEGG pathways overrepresented in genes that are up- and
down-regulated in senescent activated HSC.

| | Up | Down |
|---|---|---|
| Probe sets (54676) | 381 | 533 |
| Known genes | 294 | 352 |
| GO Annotated genes | 269 | 318 |
| GO BP | Immune response (28) p = 2.84E−10 | cell cycle (58) p = 1.72E−21 |
| | angiogenesis (11) p = 6.84E−6 | cell adhesion (30) p = 1.22E−6 |
| | chromatin assembly (10) p = 2.38E−5 | multicellular organismal development (65) p = 2.9E−8 |
| | cell differentiation (46) p = 4.77E−5 | integrin-mediated signaling (7) p = 3.76E−5 |
| | regulation of cell proliferation (20) p = 2.18E−5 | |
| | homeostasis (17) p = 3.44E−5 | |
| GO CC | extracellular region (40) p = 4.18E−12 | cytoskeleton (40) p = 3.96E−12 |
| | nucleosome (10) p = 2.79E−6 | extracellular matrix (17) 3.44E−6 |
| | | chromosome, pericentric region (12) p = 1.24E−9 |
| | | integrin complex (6) p = 5.58E−6 |
| | | cell cortex (7) p = 5.23E−5 |
| GO MF | cytokine activity (20) p = 1.98E−10 | cytoskeletal protein binding (23) p = 1.06E−7 |
| | plasminogen activator activity (3) p = 2.7E−5 | ECM structural constituent (10) p = 1.75E−6 |
| | | serine/treonine kinase activity (19) p = 5E−5 |
| KEGG pathway | Cytokine-cytokine receptor interaction (17) 6.25E−4 | ECM-receptor interaction (12) p = 5.86E−7 |
| | | focal adhesion (15) p = 3.56E−5 |
| | | cell cycle (10) p = 2.32E−4 |
| | | regulation of actin cytoskeleton (14) p = 2.29E−4 |

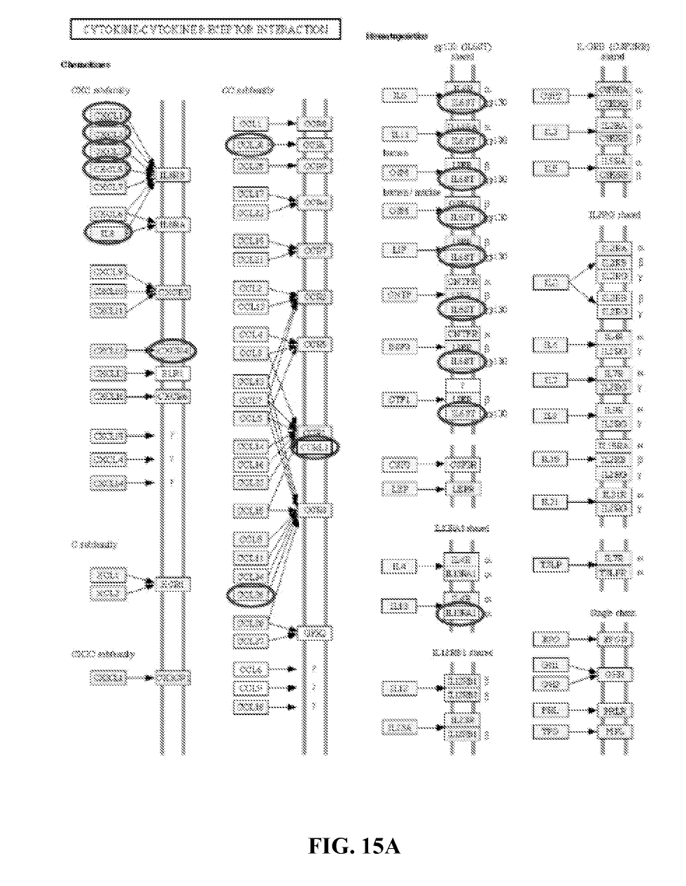

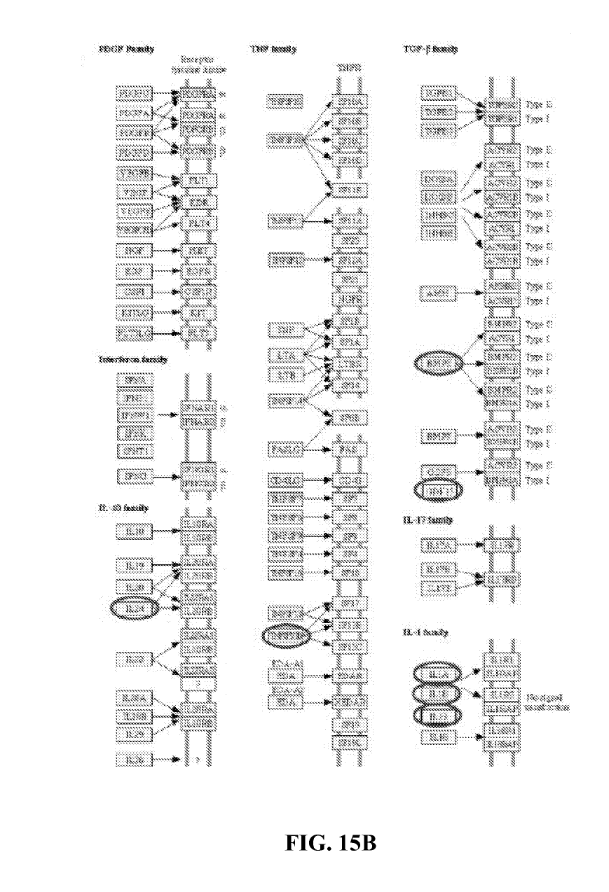

Senescent human fibroblasts also show a pattern of gene expression that involves upregulation of secreted proteases, protease modulators, growth factors and cytokines, often referred to as the “senescence-associated secretory phenotype” (Campisi and d'Adda di Fagagna, 2007, full-cite supra, the contents of which are hereby incorporated by reference). Similarly, senescent activated HSCs upregulate matrix metalloproteinases, which have fibrolytic activity (FIG. 5C). Moreover, these cells upregulated genes related to “extracellular region” and “cytokine activity” (p=4.18E-12, p=1.98E-10 respectively). The most significantly overrepresented Biological Process term among up-regulated genes was “immune response” (p=2.84E-10) and, accordingly, the only overrepresented KEGG pathway among up-regulated genes was “Cytokine-cytokine receptor interaction” (p=6.24E-4, Table 1, FIG. 15).

Many of the genes upregulated in senescent activated HSCs encoded cytokines or receptors that potentiate natural killer (NK) cell function. For example, as confirmed by RT-QPCR, MICA, the ligand of the NK cell receptor NKG2D was up-regulated in senescent activated HSCs as well as IMR-90 cells triggered to senesce by replicative exhaustion, expression of oncogenic Ras, or etoposide treatment (FIG. 5D). Additionally, the cytokine IL-8, the NKG2D receptor ligand ULBP2, and the adhesion molecule CD58(which mediates NK-target cell interactions), were also upregulated in both senescent activated HSCs and IMR-90 cells (FIG. 5D). The fact these genes were upregulated in IMR-90 cells indicates that NK cell function may also be important for eliminating senescent cells in other tissues. Thus, senescent cells (such as senescent activated HSCs) upregulate genes predicted to enhance immune surveillance.

### Example 6

### Immune Cells are Found in Proximity to Senescent Cells in Fibrotic Livers

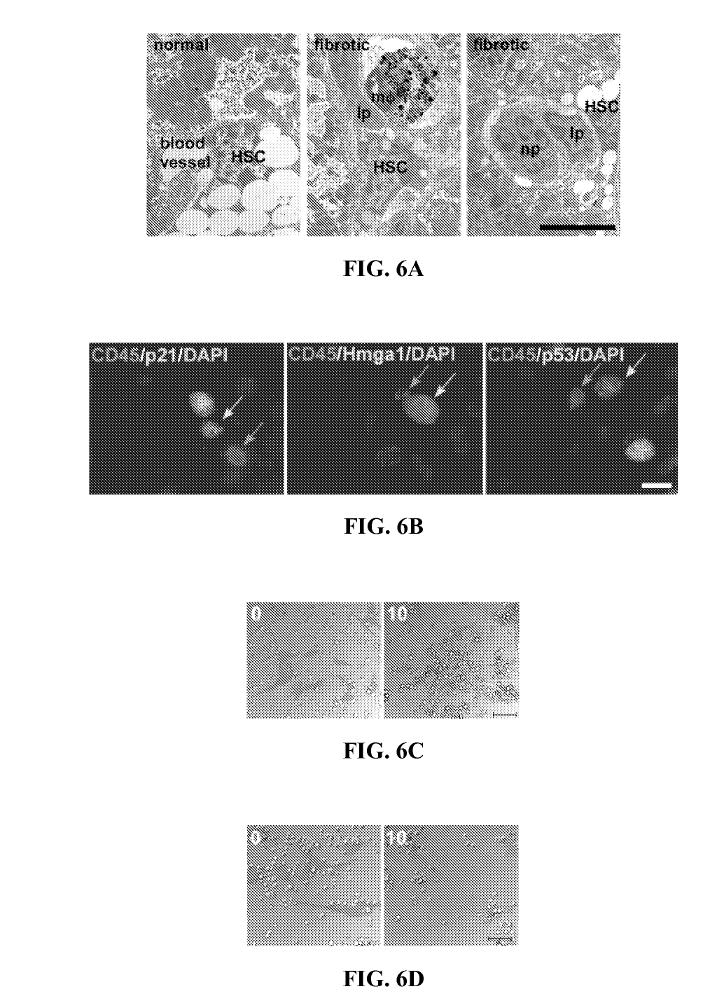

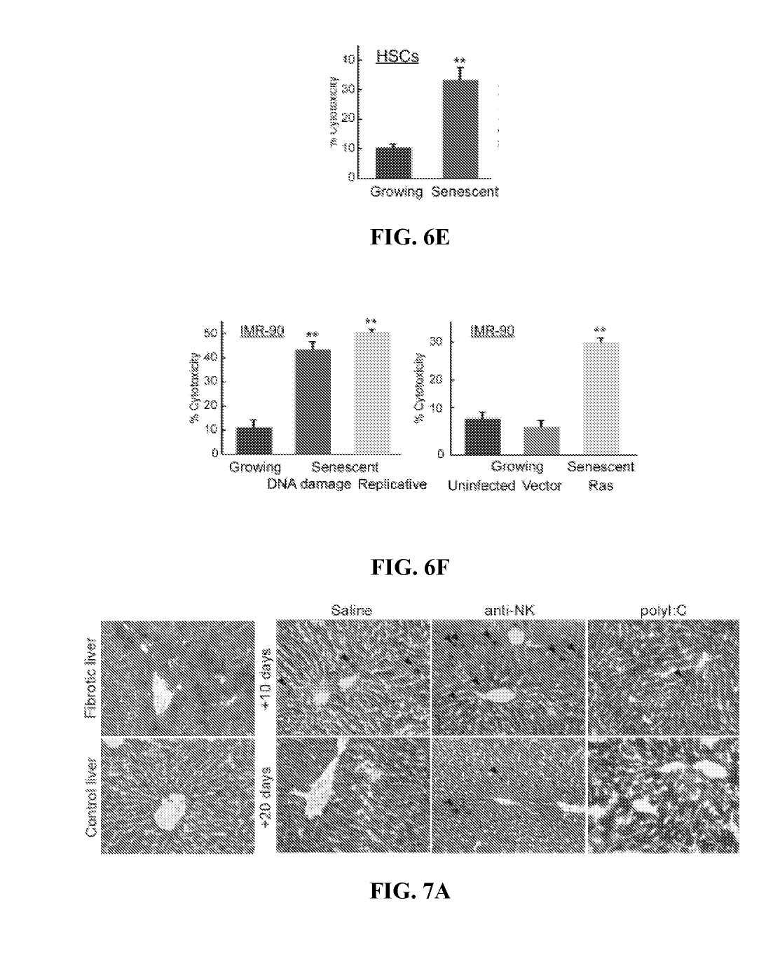

The data described above raise the possibility that senescence might limit liver fibrosis (and fibrosis generally) by downmodulating extracellular matrix production, upregulating extracellular matrix degrading enzymes and stimulating immune clearance of activated HSCs (or other cells contributing to fibrosis). NK cells are a major component of the innate immune system that recognize tumors, viruses and MHC mismatched bone marrow grafts (Raulet and Vance, 2006, Nat. Rev. Immunol., 6, 520-531, which is hereby incorporated by reference). These cells can directly lyse target cells and influence killing by components of adaptive immune system, including T-cells (Raulet and Vance, 2006). During liver cirrhosis, NK cells and other immune cell types migrate into the fibrotic scar, creating an inflammatory environment (Bataller and Brenner, 2005; Muhanna et al., 2007, Clin. Exp. Immunol., 148, 338-347, which is hereby incorporated by reference). Accordingly, an accumulation of various immune cells in fibrotic livers was observed by flow cytometry (data not shown). Using electron microscopy to identify cells by morphological characteristics together with immunofluorescence-based immunophenotyping, we observed activated lymphocytes (including NK cells), macrophages, and neutrophils adjacent to HSCs in fibrotic liver tissue from CCl4 treated mice but not normal controls (FIG. 6A). These immune cells were typically in close proximity to cells expressing the senescent markers p53, p21 and Hmga1 (FIG. 6B). These data, together with our expression analyses, raise the possibility that senescent cells produce signals that attract immune cells into fibrotic lesions.

### Example 7

### Senescent Stellate Cells are Selectively Targeted by NK cells

NK cells can be required for the clearance of senescent tumor cells in vivo (Xue et al., 2007). As senescent activated HSCs and IMR-90 cells were found to express all of the components necessary for NK cell recognition, it was tested whether they could be selectively killed by NK cells in vitro and in vivo. In initial experiments, IMR-90 cells were used since they are easily obtained. Growing and senescent IMR-90 cells were co-cultured with the NK cells at 1:10 target:effector cell ratio, and cell viability was monitored by time-lapse microscopy and quantified at 12 hours. As a source of NK cells, the line YT was used, which exhibits an NK cell immunophenotype and recognition abilities (Drexler and Matsuo, 2000, Leukemia, 14, 777-782).

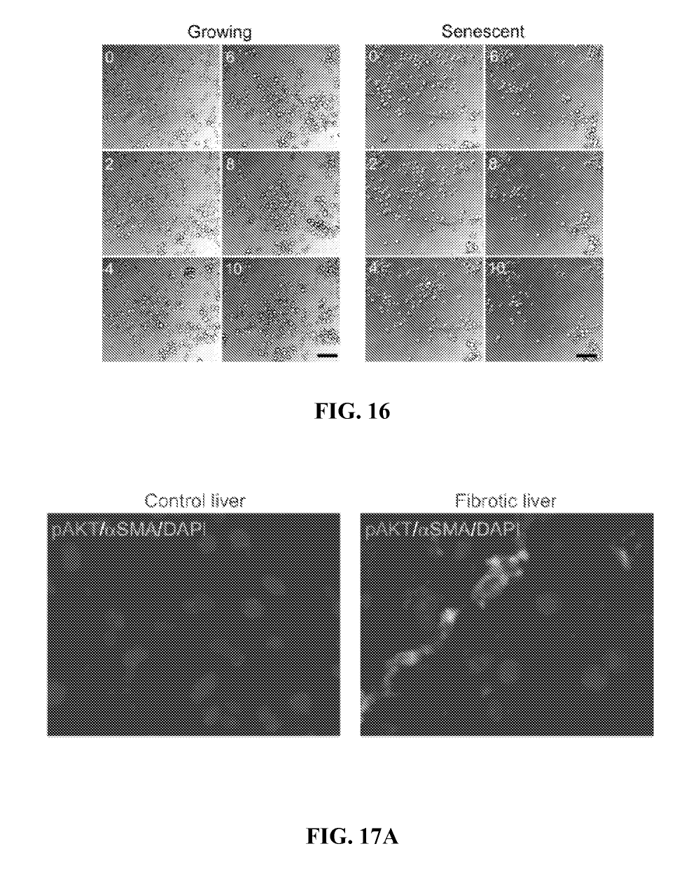

Senescent IMR-90 cells were markedly more sensitive to NK cell-mediated killing compared to growing cells. Thus, growing cells were not attacked by YT cells under these co-culture conditions and remained attached to the culture dish (FIG. 6C, FIG. 16). By contrast, senescent cells readily attracted YT cells, then underwent apoptosis and detached from the surface of the dish (FIG. 6D, FIG. 16).

YT cells were next tested as to whether they exhibit cytotoxic activity towards senescent activated HSCs by a quantitative in vitro cytotoxicity assay. In these studies, activated human HSCs were made senescent using etoposide treatment and compared to IMR-90 cells that were triggered to senesce by etoposide, replicative exhaustion, or oncogenic ras (Narita et al., 2003; Serrano et al., 1997). As assessed by crystal violet staining of cell populations at 12 hours, senescent cells were much more sensitive to NK-mediated killing (FIGS. 6E,F, p=0.0007 for activated HSCs and p=0.0002, p=0.0008, p=0.001 for etoposide, replicative exhaustion, or oncogenic ras induced cells respectively). Although this selective effect could be overcome at higher NK cell concentrations (data not shown), these cells can preferentially attack senescent cells in vitro.

To determine whether NK cells can target senescent cells in vivo and their impact on liver fibrosis, modulating NK cell function was tested for how it would influence the frequency of senescent activated HSCs and fibrosis resolution in livers obtained from mice following a six week course of CCl4 or at various times after ceasing treatment. To deplete NK cells mice were treated with neutralizing antibodies [anti-AsialoGM1 (Radaeva et al., 2006; Xue et al., 2007)] during the period following CCl4 withdrawal. Conversely, to enhance the immune response, we treated mice with polyinosinic-polycytidylic acid (polyI:C), which induces interferon-γ and enhances NK cell activity in the liver (Radaeva et al., 2006).

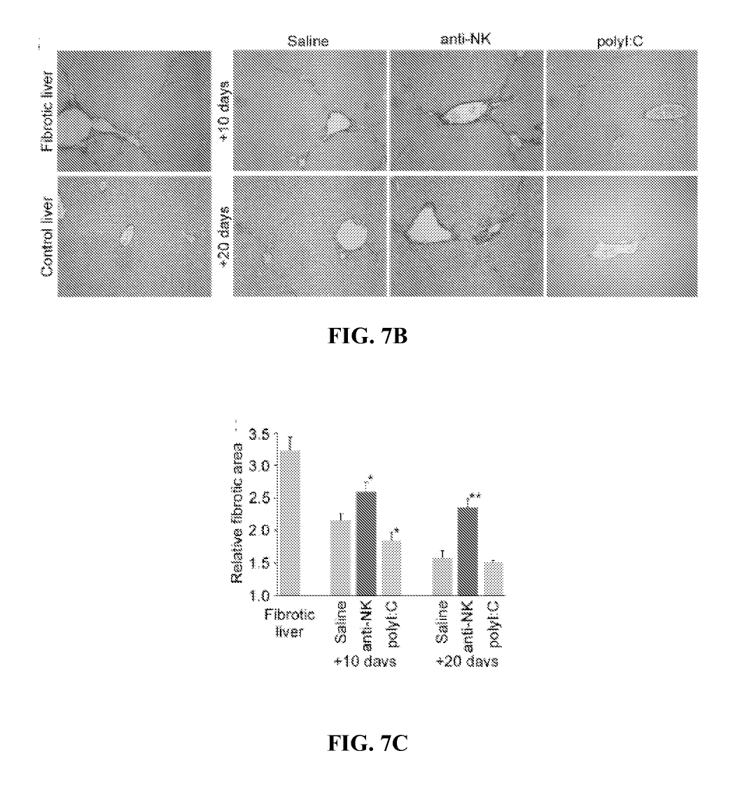

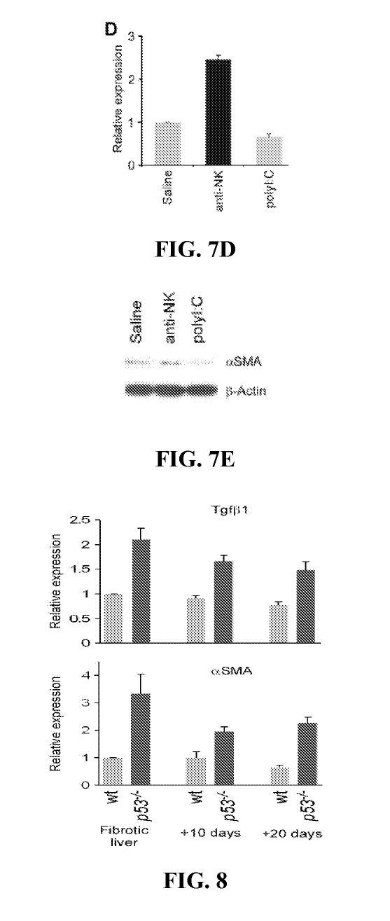

NK cell activity had a dramatic effect on the clearance of senescent cells and resolution of fibrosis. Hence, livers derived from mice treated with the anti-NK antibody retained many senescent cells and displayed significantly more fibrosis compared to saline or isotype IgG treated controls (FIGS. 7A-C; data not shown). Conversely, livers from mice treated with polyI:C for 10 or 20 days contained fewer senescent cells and less fibrotic tissue compared to controls. These changes correlated with the number of activated HSCs present, since αSMA mRNA and protein levels were increased following anti-NK antibody treatment and decreased following polyI:C treatment (FIGS. 7D,E). Therefore, the immune system can effectively eliminate senescent cells from fibrotic tissue and thereby contribute to the resolution of fibrosis.

### Example 8

### Perforin is Important for NK Mediated Killing of Senescent Cells

To kill the target cell, NK cells can use either a death receptor mediated pathway or granule exocytosis involving activity of Perforin and Granzyme proteins. It is shown below that a Perforin mediated pathway is essential for NK-mediated senescent cell killing in vitro and for defense against fibrosis in vivo.

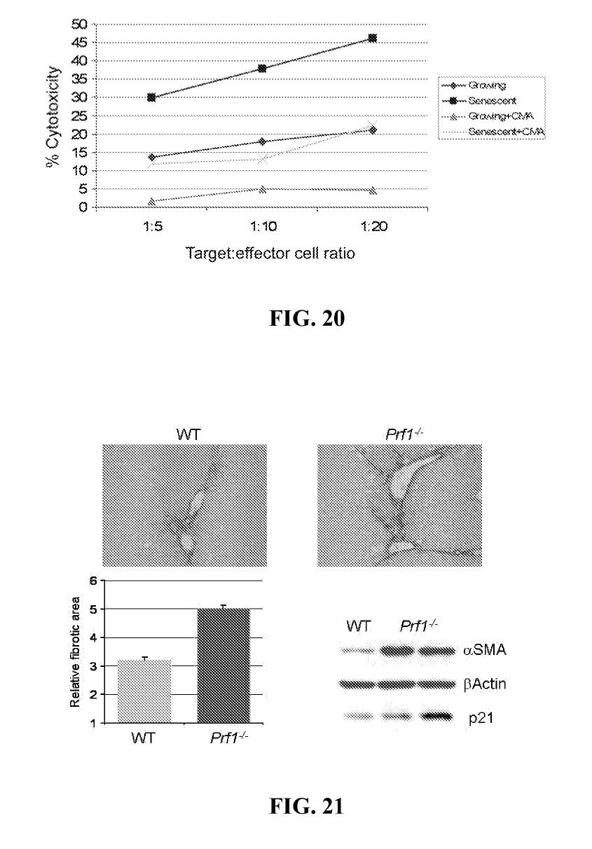

To test if a Perforin mediated pathway is involved in senescent cell killing in vitro, the in vitro killing assays of growing and senescent IMR-90 cells were used (the assay is described at Krizhanovsky, V. et al., “Senescence of Activated Stellate Cells Limits Liver Fibrosis,” Cell, 134, 657-667 (Aug. 22, 2008), which is hereby incorporated by reference). Growing and senescent IMR-90 cells were incubated with different amounts of YT cells (NK cell line) in presence or absence of granule exocytosis pathway inhibitor Concanamycin A (CMA). CMA inhibits Perforin based cytotoxic activity by accelerated degradation of Perforin by an increase in the pH of lytic granules. In absence of CMA, NK cells can preferentially kill senescent cells at wide range of target:effector cell ratios (FIG. 20). This effect was significantly inhibited in presence of CMA. Therefore, Perforin mediated cytotoxic activity is important for NK cell cytotoxicity towards senescent cells.

To study the role of Perforin mediated cytotoxicity in vivo, fibrosis was induced in wild type (WT) and Perforin knock-out (Prfl^{-/-}) mice. Fibrosis was induced by 12 consecutive intraperitoneal injections of CCl4. Prfl^{-/-} mice developed significantly stronger fibrosis then WT mice (FIG. 21). Moreover, the amount of activated stellate cells was significantly higher in the liver Prfl^{-/-} mice, as evaluated by expression of activated stellate cell marker αSMA (FIG. 21). Expression of senescence marker p21 in the liver was higher in Prfl^{-/-} mice, indicating higher amount of senescent cells in the liver of the knock-out animals. These data indicates that lack of Perforin mediated cytotoxicity in vivo leads to retention of senescent cells in fibrotic liver and stronger fibrosis. Therefore, Perforin mediated cytotoxicity is important and perhaps necessary for efficient protection against fibrosis.

What is claimed:

1. A method for treating fibrosis in a subject, the method comprising administering to the subject one or more agents in an amount sufficient to cause an increase in the number of activated innate immune cells in the fibrotic tissue and an increase in the killing of senescent cells in the fibrotic tissue.

2. The method of claim 1, wherein the fibrosis is present in the liver, lung, atherosclerotic tissue, skin, pancreas, or prostate of the subject.

3. The method of claim 1, wherein the agent(s) are administered in an amount sufficient to cause an increase in the number of activated NK cells in the fibrotic tissue.

4. The method of claim 1, wherein the agent(s) comprise one or more of IFN-α, IFN-γ, IL-1, IL-2, IL-6, IL-8, IL-13, IL-15, IL-18, IL-24, BMP2, GDF15, CXCL1, CXCL2, CXCL3, CXCL5, CXCL12, CCL20, CCL15, CCL26, LIF, CNTF, BSF3, CTF1, an agonist of NKp30, an agonist of NKp44, an agonist of NKp46, an agonist of an NKG2D receptor, an agonist of a SLAM-related receptors (SRR), and an agonist of CD48.

5. A method for treating fibrosis in a subject, the method comprises administering to the subject allogeneic NK cells activated and expanded ex vivo in an amount sufficient to cause an increase in the killing of senescent cells in the fibrotic tissue.

6. A method for treating fibrosis in a subject, the method comprising:
(a) administering to the subject one or more agents that promotes the senescence of myofibroblasts in the fibrotic tissue, and
(b) administering to the subject one or more agents that promotes the killing of the senescent myofibroblasts in the fibrotic tissue.

7. The method of claim 6, wherein the agent that promotes the senescence of myofibroblasts in the fibrotic tissue comprises an expression vector that encodes p53, p21/Cip1/Waf1 cyclin-dependent kinase inhibitor, or a miR-34 class of microRNA.

8. The method of claim 6, wherein the fibrosis occurs in the liver of the subject, and wherein the expression vector comprises a GFAP promoter.

9. The method of claim 6, wherein the agent(s) that promotes the senescence of myofibroblasts in the fibrotic tissue comprises an expression vector that codes for a dsRNA or a short-hairpin RNA molecule that can cause post-transcriptional silencing of cyclin-dependent kinases 2 and/or 4 via RNA interference.

10. The method of claim 6, wherein the agent(s) that promotes the killing of senescent myofibroblasts comprises an immunostimulatory molecule capable of activating and/or recruiting an innate immune system cell in/to the fibrotic tissue.

11. The method of claim 10, wherein the agent(s) comprise an immunostimulatory molecule capable of activating NK cells and/or recruiting NK cells to the fibrotic tissue.

12. The method of claim 11, wherein the immunostimulatory molecule comprises an agonist of NKp30, NKp44, NKp46, NKG2D receptors, or an agonist of SLAM-related receptors (SRR).

13. The method of claim 6, wherein the agent(s) that promotes the killing of senescent myofibroblast comprises an antibody that binds to one or more cell surface proteins upregulated on the senescent myofibroblast as compared to the non-senescent myofibroblast.

14. The method of claim 13, wherein the cell surface protein(s) comprise ligands of NK activation receptors (including ligands of NKp30, NKp44, NKp46, NKG2D receptors) ULBP2, PVR, and CD58.

15. The method of claim 14, wherein a ligand of NKG2D receptor is MICA.

16. A method for treating liver fibrosis, the method comprising:
(a) increasing the senescence of activated hepatic stellate cells in liver, and
(b) increasing the killing of senescent activated hepatic stellate cells.

17. A method of screening for a compound for treating fibrosis, the method comprising:
(a) providing a culture comprising:
(1) growing myofibroblast cells,
(2) senescent myofibroblast cells, and
(3) NK cells; and
(b) testing whether the addition of a compound causes a specific increase in the death of senescent myofibroblast cells, wherein the increase in the death of senescent cells is not specific if the addition of the compound also causes an increase in the death of growing myofibroblast cells and/or an increase in the death of NK cells.

18. The method of claim 17, wherein step (b) further comprises testing whether the addition of the compound causes a specific increase in the death of senescent cells that is NK-cell dependent, wherein an increase in the death of senescent cells is not NK-cell dependent if the addition of the compound causes a specific increase in the death of senescent cells in a culture that does not contain NK cells.
